(19) United States
(12) Patent Application Publication
(10) Pub. No.: US 2023/0045629 A1
(43) Pub. Date: Feb. 9, 2023

(54) TREATMENT OF PROGRESSIVE SUPRANUCLEAR PALSY

(71) Applicant: NATIONAL UNIVERSITY CORPORATION TOKAI NATIONAL HIGHER EDUCATION AND RESEARCH SYSTEM, Nagoya-shi (JP)

(72) Inventors: Masahisa KATSUNO, Nagoya-shi (JP); Koyo TSUJIKAWA, Nagoya-shi (JP); Kentaro SAHASHI, Nagoya-shi (JP)

(73) Assignee: NATIONAL UNIVERSITY CORPORATION TOKAI NATIONAL HIGHER EDUCATION AND RESEARCH SYSTEM, Nagoya-shi (JP)

(21) Appl. No.: 17/758,841

(22) PCT Filed: Jan. 14, 2021
(86) PCT No.: PCT/JP2021/001026

§ 371 Date: Jul. 14, 2022

(30) Foreign Application Priority Data
Jan. 15, 2020 (JP) 2020-004738

(51) Int. Cl. A61K 49/00; C12N 15/113; A61P 25/28

(52) U.S. Cl. CPC A61K 49/0008; C12N 15/113; A61P 25/28; C12N 2310/12; C12N 2310/14; C12N 2310/11

(57) ABSTRACT

Therapeutic methods and medicines may be developed by identifying a gene responsible for progressive supranuclear palsy, as may effective therapeutic methods and medicines. A medicine for progressive supranuclear palsy may contain a compound for inhibiting the expression of a filamin-A gene is provided. Also provided is an assessment system that uses cells expressing filamin-A, which is used in the search for medicaments for progressive supranuclear palsy or their candidates.

### TECHNICAL FIELD

The present invention relates to the treatment of progressive supranuclear palsy (PSP). More specifically, the present invention relates to medicaments for PSP and research tools for medicaments for PSP (drug assessment system).

### BACKGROUND ART

PSP is a neurodegenerative disease pathologically characterized by abnormal aggregation of tau protein in neurons and glial cells such as astroglia. The clinical manifestations of PSP vary from case to case, ranging from cases mainly with motor symptoms, such as classic Richardson's syndrome and Parkinson's syndrome, to cases mainly with psychiatric symptoms, such as frontotemporal dementia (NPL 1). Because there is no curative treatment for PSP, all patients develop progressive symptoms, and many of them die in 5 to 10 years after onset (NPL 1 and 2). Additionally, the motor and psychiatric symptoms of PSP impose a significant care burden on the family, and this is a social problem that must be resolved (NPL 3). Human tau protein is broadly classified into two isoforms, 3-repeat tau protein (3R-tau) and 4-repeat tau protein (4R-tau), depending on the number of repeats in the microtubule-binding domain. Aggregated tau protein induces cell death due to its toxicity. Diseases involving the aggregation of tau protein are referred to as “tauopathies,” and include multiple diseases such as PSP and Alzheimer's disease (AD). While AD involves the aggregation of both 3R-tau and 4R-tau, aggregation of 4R-tau is dominant in PSP. Unlike Alzheimer's disease, the lesions of PSP are predominantly distributed in the basal ganglia, midbrain tegmentum, and frontal lobe, and globose-type neurofibrillary tangle (globose-type NFT) and tufted astroglia (TA) are 4R-tau aggregate forms characteristic of PSP (NPL 1 and 4). The interaction between fused in sarcoma (FUS) and splicing factor proline- and glutamine-rich (SFPQ) RNA proteins is known to regulate the balance between 3R-tau and 4R-tau, and its failure is implicated in the pathology of tauopathies (NPL 5). However, the pathogenetic mechanism of tau protein aggregation is still unknown, and no animal model of PSP has been established.

Although PSP usually occurs in sporadic form, very rare familial forms of PSP have been recognized (NPL 1 and 6). Some familial PSP cases have mutations in the MAPT gene encoding tau protein, and the mutant tau proteins have a high aggregation propensity (NPL 1).

### CITATION LIST

### Non-Patent Literature

NPL 1: Boxer A L, Yu J T, Golbe L I, Litvan I, Lang A E, Hoglinger G U. Advances in progressive supranuclear palsy: new diagnostic criteria, biomarkers, and therapeutic approaches. Lancet Neurol 2017; 16: 552-63.
NPL 2: Glasmacher S A, Leigh P N, Saha R A. Predictors of survival in progressive supranuclear palsy and multiple system atrophy: a systematic review and meta-analysis. J Neurol Neurosurg Psychiatry 2017; 88: 402-11.
NPL 3: Uttl B, Santacruz P, Litvan I, Grafman J. Caregiving in progressive supranuclear palsy. Neurology 1998; 51: 1303-1309.
NPL 4: Yoshida M. Astrocytic inclusions in progressive supranuclear palsy and corticobasal degeneration. Neuropathology 2014; 34: 555-70.
NPL 5: Ishigaki S, Fujioka Y, Okada Y, Riku Y, Udagawa T, Honda D, et al. Altered Tau Isoform Ratio Caused by Loss of FUS and SFPQ Function Leads to FTLD-like Phenotypes. Cell Rep 2017; 18: 1118-31
NPL 6: Fujioka S, Algom A A, Murray M E, Strongosky A, Soto-Ortolaza A I, Rademakers R, et al. Similarities between familial and sporadic autopsy-proven progressive supranuclear palsy. Neurology 2013; 80: 2076-8

### SUMMARY OF INVENTION

### Technical Problem

Because some familial PSP cases do not involve any mutation in the MAPT gene, there could be genetic factors that affect the aggregation of tau protein other than the MAPT gene. The cause of PSP is unknown, and there is no effective therapeutic method or medicament. Aiming for a breakthrough in the current situation, an object of the present invention is to identify the gene responsible for PSP and to create effective therapeutic methods and medicaments. Another object is to provide a useful means for the development of therapeutic methods and medicaments.

### Solution to Problem

Study was conducted to achieve the objects, and filamin-A (FLNA) was identified as a candidate for responsible genes by neuropathological analysis and DNA microarray analysis of PSP patients. Further research provided evidence of support for filamin-A being involved in the onset or pathology of PSP, and thus filamin-A being a potential therapeutic target, and also revealed that inhibiting the expression of the filamin-A gene can have a therapeutic effect. On the basis of these findings, the following subject matter is mainly provided.

[1] A medicament for progressive supranuclear palsy, comprising a compound for inhibiting expression of the filamin-A gene.
[1A] An inhibitor for expression of 4-repeat tau, comprising a compound for inhibiting expression of the filamin-A gene.
[1B] An inhibitor for phosphorylated 4-repeat tau, comprising a compound for inhibiting expression of the filamin-A gene.
[1C] An inhibitor for aggregation of 4-repeat tau, comprising a compound for inhibiting expression of the filamin-A gene.
[2] The medicament for progressive supranuclear palsy according to [1], wherein the compound is selected from the group consisting of the following (a) to (e):
(a) an siRNA targeting the filamin-A gene;
(b) a nucleic acid construct intracellularly forming an siRNA targeting the filamin-A gene;
(c) a single-stranded RNA containing an expression suppression sequence inhibiting expression of the filamin-A gene and a complementary sequence annealing to the expression suppression sequence;
(d) an antisense nucleic acid targeting a transcript of the filamin-A gene; and
(e) a ribozyme targeting a transcript of the filamin-A gene.
[2A] The medicament for progressive supranuclear palsy according to [1] or [2], wherein the medicament is administered to a subject with an increased expression level of filamin-A. and/or 4-repeat tau in a neuron and/or a glial cell.
[2B] The medicament for progressive supranuclear palsy according to [1] or [2], wherein the medicament is administered to a subject with an expression level of 4-repeat tau higher than an expression level of 3-repeat tau in a neuron and/or a glial cell.
[3] A method for treating progressive supranuclear palsy in a subject, comprising the step of administering the medicament for progressive supranuclear palsy of [1] or [2] to a subject.
[3A] The method for treating progressive supranuclear palsy according to [3], wherein the subject has an increased expression level of filamin-A and/or 4-repeat tau in a neuron and/or a glial cell.
[3B] The method for treating progressive supranuclear palsy according to [3], wherein the subject has an expression level of 4-repeat tau higher than an expression level of 3-repeat tau in a neuron and/or a glial cell.
[4] A method for assessing efficacy of a test substance on progressive supranuclear palsy, the method comprising the following steps (i) and (ii):
(i) the step of bringing a test substance into contact with a cell expressing filamin-A; and
(ii) the step of detecting an expression of filamin-A, an amount of 4-repeat tau, and/or an amount of phosphorylated tau in the cell to determine efficacy of the test substance based on detection results, wherein a decreased expression level of filamin-A, a decreased amount of 4-repeat tau, and/or a decreased amount of phosphorylated tau is an indicator of efficacy of the test substance.
[5] The assessment method according to [4], wherein the cell is a lymphocyte cell line derived from a patient with progressive supranuclear palsy.
[6] A lymphocyte cell line derived from a patient with progressive supranuclear palsy, wherein the lymphocyte cell line has an increased expression level of filamin-A.
[7] A non-human mammal having a high expression level of filamin-A due to introduction of a filamin-A gene and presenting a progressive supranuclear palsy-like pathology.
[8] The non-human mammal according to [7], which is a transgenic animal.
[9] The non-human mammal according to [7] or [8], wherein the progressive supranuclear palsy-like pathology is increased 4-repeat tau and/or increased phosphorylated tau in a neuron and/or a glial cell.
[10] The non-human mammal according to any one of [7] to [9], wherein the non-human mammal belongs to a species (genus) selected from the group consisting of mice, rats, guinea pigs, hamsters, rabbits, dogs, cats, and monkeys.
[11] The non-human mammal according to any one of [7] to [9], wherein the non-human mammal belongs to a species (genus) of mice.
[12] A biomarker for progressive supranuclear palsy, comprising filamin-A.

### BRIEF DESCRIPTION OF DRAWINGS

FIG. 1: Japanese identical twins who developed PSP at the same time. (a): A family tree shows non-affected individuals (white) and identical twins with PSP (black, Twin-A and Twin-B). A circle indicates a female, and a square indicates a male. All 12 haplotypes found by microsatellite markers matched perfectly, and the results were consistent with the fact that Twin-A and Twin-B were identical twins. (b to n): The neuropathological findings in Twin-A and Twin-B were consistent with PSP. The frontal lobe of Twin-B was atrophic (b). In the coronal section, the internal globus pallidus (c, arrow) and the subthalamic nucleus (c, arrowhead) were atrophic. In the midbrain, the tegmentum was atrophic, and the substantia nigra showed brownish discoloration (d, arrow). Microscopic findings include aggregates of 4-repeat-tau-specific antibody RD4 positive (e) and 3-repeat-tau-specific antibody RD3 positive (f) in the globus pallidus of Twin-B at low magnification. High-magnification images for Twin-A (g to j) and Twin-B (k to n) are also shown. TA characteristic of PSP (g to i and k to m) and globose-type NFT (j to n) are shown. The scale bars are 10 mm (c and d), 20 μm (e and f), and 10 μm (g to n) in length. The photographs show Gallyas-Braak (G-B) staining (g and k), RD4 antibody staining (e, l, j, m, and n), RD3 antibody staining (f), and AT8 antibody staining (h and 1).

FIG. 2: In the identical twins who developed PSP at the same time, filamin-A (FLNA) gene duplication was identified. (a): Whole exome analysis using an eXome Hidden Markov Model (XHMM) or chromosome microarray shows that Twin-A, Twin-B, and non-affected sibling female (II-3) have about 0.3 Mb of a region with an increased number of copies at Xq28. The top row is Twin-A's XHMM. The vertical axis indicates a Z-score. The others are the microarrays of Twin-A, Twin-B, and non-affected siblings (II-1, II-2, and II-3). The vertical axis indicates the log 2 ratio. (b) A magnified view of the region with an increased number of copies recognized by the microarray in Twin-A. The interior of the dotted square in (a) is shown. The figure shows the positions of low-copy repeats (LCR) and coding genes. The region with an increased number of copies contained 16 coding genes, and the number of copies had a stepwise variation in the LCRs. The number of copies calculated by microarrays were plotted, and the FLNA gene was duplicated in two copies. (c): X chromosome inactivation (XCI) analysis using the methylated region of the FRAXA gene indicated that non-affected sibling female (II-3) had a markedly skewed pattern (XCI ratio=93:7). (d): Real-time quantitative PCR using cDNA derived from immortalized lymphocytes of Twin-A, Twin-B, and non-affected sibling female (II-3). The mRNA expression levels of the genes in the region with an increased number of copies, including the FLNA gene, were increased in Twin-A and Twin-B, but not in 11-3. The values were normalized by a housekeeping gene GUSB, and are values relative to II-1 as a control case.

FIG. 3-1: Filamin-A promotes aggregation of 4R-tau. (a): Western blotting using the frontal lobes of autopsy brains. Of the 16 coding genes in the region with an increased number of copies at Xq28, five genes (t; filamin-A (FLNA), RPL10, GDI1, FAM3A, and G6PD) in both Twin-A and Twin-B showed a value higher than the median+standard deviation of a healthy control group (Normal-1 to Normal-5). GAPDH was used as a loading control.

FIG. 3-2: continued from FIG. 3. (b): Filamin-A, among the five genes, which was co-expressed with GFP-tagged 4R-tau (GFP-4R-tau) in HEK293 cells, showed a statistically significant increase in the expression level of GFP-4R-tau compared to the empty expression, which is a control (P<0.001, n=5). A Tukey-Kramer test was performed. The multiple asterisks “***” indicate P<0.001. The error bars indicate the standard error of the mean.

FIG. 3-3: Continued from FIG. 3. (c): Western blotting of immortalized lymphocytes. Twin-A and Twin-B showed increased expression levels of filamin-A and endogenous tau protein compared with non-affected siblings (II-1, 11-2, and 11-3). The tau protein was dephosphorylated by protein phosphatase and analyzed by using TAU-5 antibody. (d): Western blotting of immortalized lymphocytes. After the treatment by using three types of siRNAs that inhibit the expression of filamin-A, the immortalized lymphocytes of Twin-A showed a decrease not only in the expression level of filamin-A but also in the expression level of endogenous 4R-tau protein. RD4 antibody was used. A Tukey-Kramer test was performed. The multiple asterisks “***” indicate P<0.001, and the single asterisk “*” indicates P<0.05. The error bars indicate the standard error of the mean.

FIG. 3-4: Continued from FIG. 3. (e): Western blotting of a TBS-soluble fraction (S1) extracted from HEK293 cells expressing filamin-A and GFP-4R-tau. The use of phosphorylated tau antibody AT8 (Ser202/Thr205) and PHF-1 (Ser396/Ser404) indicated increased phosphorylation of GFP-4R-tau by the expression of filamin-A. (f): A cycloheximide (CHX) chase experiment confirmed the protein stability of GFP-4R-tau by the expression of filamin-A (n=3). CHX was added to HEK293 cells expressing filamin-A and GFP-4R-tau, and proteins were collected at the indicated time points (n=3).

FIG. 3-5: Continued from FIG. 3. (g) Western blotting performed using a homogenate (Ho), a TBS-soluble fraction (S1), and a sarkosyl-insoluble fraction (P3) of HEK293 cells expressing filamin-A and GFP-4R-tau. In this experiment, the cells were transfected with various amounts of a plasmid of filamin-A as shown in the figure. In Ho, the expression level of GFP-4R-tau was increased dependently on the expression level of filamin-A (n=3). In S1 and P3, the expression level of GFP-4R-tau was statistically significantly increased at the highest expression level of filamin-A (S1 and P3 both: P<0.05). The arrows indicate GFP-4R-tau, and the arrowheads indicate endogenous tau.

FIG. 3-6: Continued from FIG. 3. (h): Coimmunoprecipitation using TAU-5 antibody. In HEK293 cells expressing filamin-A and GFP-4R-tau, filamin-A, heat shock proteins HSP90, HSP70, and HSP40, and ubiquitin were immunoprecipitated together with GFP-4R-tau.

FIG. 4-1: Filamin-A is colocalized with aggregated tau in autopsied PSP brain, and an experimentally excessive amount of filamin-A induces tau aggregation in the primary astroglia. (a): Western blotting of the frontal lobes of 34 cases. The TBS-soluble fraction (S1) and sarkosyl-insoluble fraction (P3) both exhibited a statistically significant increase in the expression level of filamin-A in the cases with PSP compared with the healthy control group and cases with neurodegenerative diseases other than PSP (S1: P<0.01, P3: P<0.05). GAPDH was used as a loading control. Dotted lines indicate membrane boundaries. A Tukey-Kramer test was performed on the parametric data of S1, and a Steel-Dwass test was performed on the nonparametric data of P3.

FIG. 4-2: Continued from FIG. 4. (b): In P3 of 11 cases of autopsied PSP brains (Twin-A, Twin-B, and 9 sporadic PSP cases), the expression level of filamin-A was positively correlated with the expression level of 4R-tau. The correlation was evaluated with the test of no correlation of Pearson product-moment correlation coefficient.

FIG. 4-3: Continued from FIG. 4. (c to e): Fluorescent immunostaining of the frontal lobes of Twin-B (c), PSP-6 (d), and PSP-9 (e). Filamin-A (original image shown in red) and phosphorylated tau AT8 (original image shown in green) were colocalized in TA or NFT. The scale bars are 5 μm.

FIG. 4-4: Continued from FIG. 4. (f): Fluorescent immunostaining of rat primary astroglia co-expressing filamin-A (original image shown in red) and GFP-4R-tau (original image shown in green). When filamin-A was expressed, GFP-4R-tau was aggregated in the cell body and proximal processes of astroglia. Next to the low-magnification photographs with dotted squares, high-magnification photographs are shown. The arrows indicate aggregated GFP-4R-tau. The scale bars are 5 μm.

FIG. 4-5: Continued from FIG. 4. (g): In western blotting of the primary astroglia, the expression level of GFP-4R-tau was statistically significantly increased when filamin-A was expressed (P<0.01, n=3). The arrowhead refers to nonspecific bands. A Student's t-test was performed. The multiple asterisks **” and single asterisk “*” indicate P<0.01 and P<0.05, respectively. The error bars indicate the standard error of the mean.

FIG. 5: Gray matter heterotopia of Twin-B. Twin-B showed gray matter heterotopia in the anterior horn of the right lateral ventricle (inside the white dotted squares: a and b) and the cerebellum (inside the solid black line square: c). The scale bars are 10 mm (a), 500 μm (b), and 1 mm (c). The microphotographs are of Kluver-Barrera staining (b and c).

FIG. 6: Neuroradiological imaging of Twin-A. (a to d): Brain MRI of Twin-A at the age of 66. Horizontal sections of a T2-weighted image showed brain atrophy mainly in the frontal lobe and the temporal lobe (a to c). A sagittal section of a T1-weighted image showed midbrain atrophy (d, arrow).

(e to g): ^{99m}Tc-ECD cerebral blood flow SEPCT images of Twin-A at the age of 66. A decreased blood flow was observed in the frontal and temporal lobes. Rt indicates the right side.

FIG. 7: Neuropathological findings in Twin-A. The cerebrum, cerebellum, and brainstem were entirely atrophied (a, b). The coronal section showed atrophy of the internal globus pallidus (c, arrows) and the subthalamic nucleus (c, arrowheads). In the midbrain, the tegmentum was atrophic, and the substantia nigra showed brownish discoloration (d, arrow). In microscopic findings, neurological deficits and gliosis were observed in the subthalamic nucleus (e), internal globus pallidus (f), midbrain tegmentum (g), and midbrain substantia nigra (h). In the midbrain substantia nigra, globose-type NFT (i) was observed. The scale bars are 5 mm (c), 10 mm (d), 50 μm (e to g), 100 μm (h), and 5 μm (i). The photographs showed hematoxylin-eosin staining (e to i).

FIG. 8: Whole exome analysis of Twin-A and healthy Japanese males using XHMM. Z scores of the Xq28 chromosomal region were extracted from XHMM data and graphed. The copy number abnormalities, including the filamin-A (FLNA) gene, found in Twin-A, were not observed in 513 healthy Japanese males.

FIG. 9: Analysis of the number of copies by real-time quantitative PCR. (a): The results of chromosome microarray of Twin-A and the location information of the primer pairs (#1 to #5. #2 is the FLNA gene region) used in real-time quantitative PCR. The lower part of the figure shows variations in the number of copies. (b): Analysis of the number of copies by real-time quantitative PCR using the genomic DNA of Twin-A and Twin-B. As with the results of microarray, the number of copies changed stepwise from 1 copy to 3 copies in a specific region of Xq28. The MECP2 gene of the same Xq28 are located outside the region with an increased number of copies and was used as a reference gene. The genomic DNA of a male sibling (II-2) of the twins was used as a control sample. (c): Analysis of the number of copies by real-time quantitative PCR using the genomic DNA of sporadic PSP (PSP-1 to PSP-9). Twin-A and Twin-B had 2 copies of the FLNA gene, but 9 cases of sporadic PSP all had 1 copy. The MECP2 gene was used as a reference gene, and the genomic DNA of PSP-1 was used as a control sample.

FIG. 10: The effect of increasing the expression level of 4R-tau by filamin-A was diminished by the introduction of filamin-A p.Ala39Gly mutation (FLNA^{Ala39Gly} FLNA^{Ala39Gly} was expressed together with GFP-4R-tau in HEK293 cells. Compared with co-expression with wild-type filamin-A (FLNA^{WT}), the expression level of GFP-4R-tau was statistically significantly decreased (P<0.05, n=3). A Student's t test was performed. The single asterisk “*” indicates P<0.05. The error bars indicate the standard error of the mean.

FIG. 11: A correlation between TBS-soluble filamin-A and the age at onset of PSP. In 11 cases of PSP, including Twin-A and Twin-B, a negative correlation was observed between TBS-soluble filamin-A and the age at onset of PSP. The correlation was evaluated by the Pearson product-moment correlation coefficient.

FIG. 12: The clinical course and neuropathological features of Twin-A and Twin-B. NFT: globose-type neurofibrillary tangle. TA: tufted astroglia. The severity of neuronal cell death/tau pathology is indicated as none (−), mild (+), moderate (++), and severe (+++).

FIG. 13: The coding genes in the region with an increased number of copies of Xq28. chrX: chromosome X, CNS: central nervous system, and MIM: Mendelian Inheritance in Man.

FIG. 14: Clinical information and neuropathological features of the autopsied brains of 34 cases. PSP: progressive supranuclear palsy; CBD: corticobasal degeneration; AD: Alzheimer's disease; PD: Parkinson's disease; DLB: dementia with Lewy bodies; ALS: amyotrophic lateral sclerosis; bvFTD: behavioral variant frontotemporal dementia; MSA: multiple system atrophy; SjS: Sjogren's syndrome; CIDP: chronic inflammatory demyelinating polyneuropathy; PE: pulmonary embolism; M: male; F: female; PMI: post-mortem interval; ND: not done; AG: argyrophilic grain; CERAD: Consortium to Establish a Registry for Alzheimer's Disease. The single asterisk “*” indicates the brain weight of a cerebral hemisphere.

FIG. 15-1: (a): Module structure. Wild-type filamin-A (FLNA^{WT}) has an N-terminal actin-binding domain (ABD) and 24 immunoglobulin-like domains (Ig). FLNA^{ABD+Ig1-15+Ig24} is truncated FLNA composed of an actin-binding domain (ABD) involved in protein interaction with F-actin, 1^{st} to 15^{th} Ig, and 24^{th} Ig involved in dimerization of FLNA. FLNA^{ABD+Ig9-15+Ig24} (ΔFLNA) is truncated FLNA composed of ABD, 9^{th} to 15^{th} Ig, and 24^{th} Ig. AAV9-ΔFLNA-6×His is an adeno-associated viral vector type 9 (AAV9) carrying ΔFLNA cDNA. CBA is a chicken β-actin promoter, 6×His is a 6×His-tag protein, WPRE is a woodchuck hepatitis virus posttranscriptional regulation element, and SpA is SV40 poly A.

FIG. 15-2: Continued from FIG. 15. (b): Immunoprecipitation using a tau antibody (TAU-5 antibody). HEK293 cells were transfected with each plasmid. ΔFLNA interacts with tau protein as with wild-type FLNA (FLNA^{WT}) and FLNA^{ABD+Ig1-15+Ig24}.

FIG. 15-3: Continued from FIG. 15. (c): Fluorescent immunostaining using a tau antibody (K9JA) and a FLNA antibody. (d): Western blotting using a 4-repeat tau antibody (RD4) and a phosphorylated tau antibody (AT8). A 2-month-old wild-type (WT) mouse was injected at the right frontal lobe with AAV9-ΔFLNA-6×His, and analyzed at the age of 3 months. The single asterisk “*” indicates the site of injection. An increase in the expression level of endogenous tau and phosphorylation of endogenous tau due to ΔFLNA was observed in the mouse. The control was a WT mouse injected with AAV9-empty-6×His.

FIG. 15-4: Continued from FIG. 15. (e): Fluorescent immunostaining using a 4-repeat tau antibody (RD4), a 3-repeat tau antibody (RD3), and a 6×His antibody. A 2-month-old genetically modified mouse (hT-PAC-N) expressing human tau protein was injected at the right frontal lobe with AAV9-ΔFLNA-6×His, and analyzed at the age of 3 months. The expression level of both 4-repeat tau and 3-repeat tau increased because of ΔFLNA. (f) Western blotting. A TBS-soluble fraction (S1) and a sarkosyl-insoluble fraction (P3) were extracted from the brain homogenate (Ho). In S1 and P3, the expression level of both 4-repeat tau and 3-repeat tau was increased. The control was hT-PAC-N injected with AAV9-empty-6×His.

FIG. 16: Immunostaining of a transgenic mouse (hFLNA-Tg) having human filamin-A (FLNA) expression induced downstream of the CAG promoter (8 months old). An increase in the expression level of FLNA and 4-repeat tau was observed in the hippocampus and the frontal cortex. The control was a non-transgenic mouse (non-Tg).

FIG. 17-1: (a): Each plasmid was introduced into a fetal mouse brain in a mouse at 14 days of gestation (E14) by in utero electroporation, and fluorescent immunostaining was performed at 18 days of gestation (E18). Wild-type FLNA (FLNA^{WT}) caused gray matter heterotopia and increased fluorescent brightness of GFP-4R-tau. However, actin-binding-lacking mutant FLNA (p.Ala39Gly mutant filamin-A: FLNA^{A39G}) did not show such changes. The single asterisk “*” and multiple asterisks “**” respectively indicate a Tukey's test P value of less than 0.05 and less than 0.01.

FIG. 17-2: Continued from FIG. 17. (b): Each plasmid was introduced into a fetal mouse brain in a mouse at 14 days of gestation (E14) by in utero electroporation, and primary cortical neurons were collected at 15 days of gestation (E15), followed by 2-day cell culture (2 DIV) and then fluorescent immunostaining. At the time of administration of 0.1% DMSO (control), the expression level of AT8-positive phosphorylated tau was increased because of wild-type FLNA (FLNA^{WT}) but not at the time of administration of an actin polymerization inhibitor cytochalasin D (CytoD). The multiple asterisks “***” indicate a Tukey's test P value of less than 0.001.

### DESCRIPTION OF EMBODIMENTS

### 1. Treatment of Progressive Supranuclear Palsy (PSP)

The first aspect of the present invention is an embodiment based on the finding that filamin-A is involved in the onset or pathology of PSP, and relates to a medicament for PSP containing a compound for inhibiting the expression of the filamin-A gene (“medical drug of the present invention” below), preferably a medicament for PSP containing a compound for inhibiting the expression of the filamin-A gene as an active ingredient. PSP is one of the neurodegenerative diseases that involve abnormal lesions of tau (tauopathies). In PSP, loss of neurons occurs, for example, in the globus pallidus, subthalamic nucleus, cerebellar dentate nucleus, red nucleus, substantia nigra, or brain-stem tegmentum, and abnormally phosphorylated tau proteins accumulate inside neurons and glial cells. The cause and pathogenic mechanism are unknown, and no effective therapeutic methods are currently available for the disease.

In the present specification, the term “medicament” refers to a medical drug that shows a therapeutic or prophylactic effect on a target disease or target pathology (i.e., PSP). The therapeutic effect includes alleviation of symptoms characteristic of a target disease and its pathology or its concomitant symptoms (i.e., decreasing the severity of the disease), and prevention or retardation of the progress of symptoms. The latter can be viewed as one of the prophylactic effects in the respect of preventing an increase in severity of diseases. Thus, the therapeutic effect and the prophylactic effect are concepts that overlap in part. A typical prophylactic effect is the prevention or retardation of the recurrence of symptoms characteristic of a target disease and its pathology. Any substance that has a therapeutic effect or prophylactic effect, or both effects, on a target disease and its pathology is considered to be a medicament for the target disease and its pathology.

Filamins are actin filament-crosslinking proteins and known to be categorized into three types of filamins: A, B, and C. Whereas filamin-A and filamin-B are expressed in various organs, filamin-C is only expressed in muscle. Filamins are a dimer of subunits with a molecular weight of about 280 kD self-associating at their C-terminus, and crosslinking actin filaments in a lattice-like fashion to form a gel structure by using the actin-binding domain at their N-terminus. Mutations to the filamin-A gene are reported as being involved in periventricular gray matter heterotopia or familial cardiac valvular dystrophy. The sequence of filamin-A isoform 1 and the sequence of the gene encoding filamin-A isoform 1 (transcript variant 1) are respectively shown in SEQ ID NO: 1 (DEFINITION: filamin-A isoform 1 [Homo sapiens]. ACCESSION: NP_001447. VERSION: NP_001447.2) and SEQ ID NO: 2 (DEFINITION: Homo sapiens filamin-A (FLNA), transcript variant 1, mRNA. ACCESSION: NM 001456. VERSION: NM 001456.3). The sequence of filamin-A isoform 2 and the sequence of the gene encoding filamin-A isoform 2 (transcript variant 2) are respectively shown in SEQ ID NO: 3 (DEFINITION: filamin-A isoform 2 [Homo sapiens]. ACCESSION: NP_001104026. VERSION: NP_001104026.1) and SEQ ID NO: 4 (DEFINITION: Homo sapiens filamin-A (FLNA), transcript variant 2, mRNA. ACCESSION: NM 001110556. VERSION: NM 001110556.2).

The compound for inhibiting the expression of the filamin-A gene is a compound that inhibits the expression process of the filamin-A gene (including transcription, posttranscriptional regulation, translation, and posttranslational regulation). The compound may be those identified by the screening described later.

In an embodiment of the present invention, the compound for inhibiting the expression of the filamin-A gene is an isolated nucleic acid. The nucleic acid may have one or more chemical modifications as described below, for example, with the aim of preventing degradation by a hydrolase such as a nuclease.

(1) A phosphoric acid residue [phosphodiester; —O—P(═O)(O⁻)—O—] of at least some nucleotides may be substituted with, for example, phosphorothioate [—O—P(═O)(S⁻)—O—], methylphosphonate [—O—P(═O)(CH₃)—O—], phosphorodithioate [—O—P(═S) (S⁻)—O—], boranophosphate [—O—P(═O)(BH₃⁻)—O—], phosphotriester [—O—P(═O)(OR)—O— (wherein R represents, for example, —CH₂CH₂CN)], or phosphoramidate [—NH—P(═O)(O⁻)—O—].
(2) In at least some nucleotides, the sugar may be substituted with morpholine, and the phosphoric acid residue may be substituted with phosphorodiamidate [—P(═O)(NR₂)—O— (wherein R represents, for example, —CH₃)].
(3) In at least some ribonucleotides, the hydroxyl group at position 2 of the sugar (ribose) may be substituted with —OR (wherein R represents, for example, —CH₃, —CH₂CH₂OCH₃, —CH₂CH₂NHC (NH) NH₂, —CH₂CONHCH₃, or —CH₂CH₂CN).
(4) In at least some nucleotides, the base (pyrimidine, purine) may be chemically modified. Examples of chemical modifications include introduction of a methyl group or a cationic functional group into position 5 of a pyrimidine base, or substitution of the carbonyl group at position 2 with thiocarbonyl.
(5) In at least some nucleotides, the phosphoric acid moiety or hydroxyl moiety may be modified with, for example, biotin, an amino group, a lower alkylamine group, or an acetyl group.
(6) In at least some ribonucleotides, the 2′oxygen and the 4′carbon of the sugar may be crosslinked to make substitution into, for example, BNA, LNA, or ENA, whose sugar conformation is locked in N form.
(7) At least some nucleotides may be substituted with a non-nucleotide, nucleic acid analog such as PNA.
(8) The nucleic acid may be conjugated with sterol such as cholesterol; vitamins such as cx-tocopherol or folate; N-acetylgalactosamine; a fatty acid; or a polymer such as polyethylene glycol, polyamine, or a cell-penetrating peptide.

Examples of compounds for inhibiting the expression of the filamin-A gene include the following. The “inhibition of expression” in the present invention can be either transient inhibition or permanent inhibition.

(a) An siRNA targeting the filamin-A gene
(b) A nucleic acid construct intracellularly forming an siRNA targeting the filamin-A gene
(c) A single-stranded RNA having an expression suppression sequence inhibiting the expression of the filamin-A gene and a complementary sequence annealing to the sequence
(d) An antisense nucleic acid targeting the transcript of the filamin-A gene
(e) A ribozyme targeting the transcript of the filamin-A gene

The compounds (a) and (b) are those used in the suppression of expression by “RNAi” (RNA interference). In other words, a medical drug containing the compound (a) or (b) of the present invention can inhibit the expression of the filamin-A gene by RNAi. RNAi is a process of sequence-specific post-transcriptional gene suppression that can be triggered in eukaryotic cells. RNAi in mammalian cells uses short double-stranded RNA (siRNA) that has a sequence corresponding to that of target mRNA. Typically, siRNA is composed of 15 base pairs or more, 16 base pairs or more, 17 base pairs or more, 18 base pairs or more, 19 base pairs or more, 20 base pairs or more, or 21 base pairs or more, and 32 base pairs or less, 31 base pairs or less, 30 base pairs or less, 29 base pairs or less, 28 base pairs or less, 27 base pairs or less, 26 base pairs or less, 25 base pairs or less, 24 base pairs or less, or 23 base pairs or less. For example, siRNA is composed of 21 to 23 base pairs. Mammalian cells are known to have two pathways (sequence-specific pathway and non-sequence-specific pathway) affected by double-stranded RNA (dsRNA). In the sequence-specific pathway, a relatively long dsRNA is split into short interfering RNAs (i.e., siRNA). On the other hand, the non-sequence-specific pathway is thought to be triggered by any dsRNA irrespective of its sequence as long as it has a predetermined length or more. In this pathway, dsRNA activates two enzymes: PKR, which becomes active and ends all protein synthesis by phosphorylating translation initiation factor eIF2, and 2′,5′oligoadenylate synthase, which is involved in the synthesis of RNAase L activation molecules. To minimize the progression of this non-sequence-specific pathway, double-stranded RNA (siRNA) of shorter than about 30 base pairs is preferable for use (see Hunter et al. (1975), J Biol Chem 250: 409-17; Manche et al. (1992), Mol Cell Biol 12: 5239-48; Minks et al. (1979), J Biol Chem 254: 10180-3; and Elbashir et al. (2001), Nature 411: 494-8).

To form target-specific RNAi, siRNA composed of sense RNA homologous to part of the mRNA sequence of the filamin-A gene (e.g., the sequence represented by SEQ ID NO: 2 or 4) and antisense RNA complementary to the sense RNA may be intracellularly introduced, or intracellularly expressed. The compound (a) can be used in the former method, and the compound (b) can be used in the latter method.

siRNA targeting the filamin-A gene is typically double-stranded RNA formed by the hybridization of sense RNA composed of a sequence homologous to a continuous region of mRNA of the gene and antisense RNA composed of a sequence complementary to the sequence. The “continuous region” is typically 15 to 30 bases, preferably 18 to 23 bases, and more preferably 19 to 21 bases in length.

Double-stranded RNA with an overhang of a few bases at a terminus is known to have a high RNAi effect. Thus, it is preferable to use siRNA with such a structure in the present invention. The base length of the overhang is not particularly limited, and the overhang is preferably 2 bases in length (e.g., TT or UU).

siRNA formed from modified RNA may be used. Examples of modifications include phosphorothioation, and the use of modified bases (e.g., fluorescently labeled bases).

In an embodiment of the present invention, the sense RNA of siRNA has a base sequence, for example, at least 80%, preferably at least 85%, more preferably at least 90%, and still more preferably at least 95% identical to the base sequence represented by any of SEQ ID NOs: 9 to 11. The “identity” of the base sequence can be calculated with default parameters (initial setting) of homology algorithm BLAST (basic local alignment search tool) of the National Center for Biotechnology Information (NCBI) in the US (http://www.ncbi.nlm.nih.gov/BLAST/).

siRNA can be designed and prepared by an ordinary method. siRNA is designed typically by using a sequence (continuous sequence) unique to a target sequence. Programs and algorithms for selecting appropriate target sequences have been developed.

The “nucleic acid construct intracellularly forming siRNA” in the compound (b) above refers to a nucleic acid molecule that, when introduced into a cell, produces desired siRNA due to intracellular processes (siRNA that causes RNAi against the filamin-A gene). One example of such nucleic acid constructs is shRNA (short-hairpin RNA). shRNA has a structure formed by sense RNA and antisense RNA linked via a loop structure (hairpin structure), and the loop structure is cleaved inside a cell to form double-stranded siRNA, which provides an RNAi effect. The loop structure can be of any length, and is typically 3 to 23 bases in length.

Another example of the nucleic acid constructs is a vector capable of expressing desired siRNA. Such vectors include vectors that express shRNA (having a sequence encoding shRNA inserted), which is converted to siRNA by a later process (“stem-loop type” or “short hairpin type”), and vectors that express sense RNA and antisense RNA separately (“tandem type”). Those skilled in the art can prepare these vectors in accordance with an ordinary method (see, for example, Brummelkamp T R et al. (2002) Science 296:550-553; Lee N S et al. (2001) Nature Biotechnology 19:500-505; Miyagishi M & Taira K (2002) Nature Biotechnology 19:497-500; Paddison P J et al. (2002) Proc. Natl. Acad. Sci. USA 99:1443-1448; Paul C P et al. (2002) Nature Biotechnology 19:505-508; Sui G et al. (2002) Proc Natl Acad Sci USA 99(8):5515-5520; and Paddison P J et al. (2002) Genes Dev. 16:948-958). A variety of vectors for RNAi are presently available. The vector of the present invention may be constructed by using such known vectors. In this case, insert DNA encoding desired RNA (e.g., shRNA) is prepared, and then the insert DNA is inserted into the cloning site of a vector to prepare RNAi expression vector (see, for example, Meng X et al. (2004) J Biol Chem 279(7):6098-6105).

The origin and structure of the vector are not limited as long as the vector has the functionality of intracellularly forming siRNA that exerts RNAi action against the filamin-A gene. Thus, various viral vectors (e.g., adenoviral vectors, adeno-associated viral vectors, retroviral vectors, lentiviral vectors, herpes virus vectors, and Sendai virus vectors), and non-viral vectors (e.g., liposomes, and positively charged liposomes) are usable. Examples of promoters usable in vectors include U6 promoter, H1 promoter, and tRNA promoter. These promoters are of RNA polymerase III, and expected to have high expression efficiency.

Single-stranded RNA of a predetermined structure is reported as being useful in the inhibition of expression of a target gene (e.g., WO2012/005368, JP2013-55913A, JP2013-138681A, and JP2013-153736A). Thus, in an embodiment of the present invention, the expression of the filamin-A gene is inhibited by using single-stranded RNA (the compound (c)) by the same mechanism as that of the inhibition of expression by siRNA (i.e. RNA interference). The single-stranded RNA of the present invention has an expression suppression sequence corresponding to the filamin-A gene and a complementary sequence capable of annealing to the sequence. The order of the linkage of the expression suppression sequence and the complementary sequence is not particularly limited. The expression suppression sequence and the complementary sequence may be linked directly or via a linker region. The linker region can be formed of a nucleotide residue or a non-nucleotide residue (e.g., the structure of polyalkylene glycol, a pyrrolidine skeleton, or a piperidine skeleton).

Examples of folios of the single-stranded RNA of the present invention include a molecule in which the 5′ region and the 3′ region anneal intramolecularly to form a single double-stranded structure (stem structure) (example 1), and a molecule in which the 5′ region and the 3′ region separately anneal intramolecularly to form two double-stranded structures (stem structure) (example 2).

An expression suppression sequence shows activity of inhibiting the expression of the filamin-A gene when the single-stranded RNA of the present invention is intracellularly introduced. Typically, a sequence that causes the suppression of expression by siRNA (i.e., RNA interference) is used as an expression suppression sequence. For example, the sequence of RNA (antisense RNA) constituting the siRNA described above (the compound (a)) can be used as an expression suppression sequence. The expression suppression sequence can be of any length, and is, for example, 18 to 32 bases, preferably 19 to 30 bases, and more preferably 19 to 21 bases in length.

The single-stranded RNA of the present invention can be of any length. The lower limit of the total number of bases that constitute the single-stranded RNA (the number of bases of the full length) is, for example, 38 bases, preferably 42 bases, more preferably 50 bases, still more preferably 51 bases, and particularly preferably 52 bases, and the upper limit is, for example, 300 bases, preferably 200 bases, more preferably 150 bases, still more preferably 100 bases, and particularly preferably 80 bases. Of the single-stranded RNA having a linker region of the present invention, the lower limit of the total number of bases excluding the linker region is, for example, 38 bases, preferably 42 bases, more preferably 50 bases, still more preferably 51 bases, and particularly preferably 52 bases, and the upper limit is, for example, 300 bases, preferably 200 bases, more preferably 150 bases, still more preferably 100 bases, and particularly preferably 80 bases.

For designing or preparing the single-stranded RNA of the present invention, reports such as the patent publications mentioned above can be referred to.

The compound (d) is a compound used in the inhibition of expression by the antisense method. In other words, a medical drug containing the compound (d) of the present invention can inhibit the expression of the filamin-A gene by the antisense method. For example, to inhibit expression by the antisense method, an antisense construct that forms RNA complementary to the unique portion of mRNA encoding the filamin-A gene is used when transcription occurs in a target cell. Such an antisense construct, for example, in the form of an expression plasmid is introduced into a target cell. Also usable is an oligonucleotide probe that hybridizes with mRNA and/or a genomic DNA sequence encoding the filamin-A gene to thereby inhibit the expression when introduced into a target cell as an antisense construct. Such an oligonucleotide probe for use is preferably resistant to endogenous nucleases such as exonucleases and/or endonucleases.

The antisense nucleic acid can be of any sequence that shows activity of inhibiting the expression of the filamin-A gene. The antisense nucleic acid may be those that bind to mRNA or its precursor (pre-mRNA) encoding the filamin-A gene to induce degradation by RNase such as RNase H, or those that bind to the splicing regulation site of pre-mRNA (e.g., an exon-intron boundary region, the region rich in purine bases in an exon) to induce exon skipping or exon inclusion. The length of the sequence of the antisense nucleic acid is, for example, 12 bases or more, 13 bases or more, 14 bases or more, or 15 bases or more, and is, for example, 50 bases or less, 45 bases or less, 40 bases or less, or 35 bases or less.

Examples of antisense nucleic acids include gapmers. Gapmers typically have a structure in which the central region (gap) is located between two terminal regions (wings), and the wings are formed of chemically modified nucleotides (e.g., nucleotides chemically modified at their sugar moiety such as BNA, LNA, ENA, or a ribonucleotide having its hydroxyl group at position 2 replaced with —OCH₃). The gap is formed of a non-chemically modified nucleotide. The phosphoric acid residue of each nucleotide is phosphorothioated. The gap can serve as a substrate for RNase. The sequence of the wings can be of any length, and is, for example, 2 bases or more, preferably 3 bases or more, and, for example, 5 bases or less in length. The length of the gap sequence is, for example, 5 bases or more, preferably 6 bases or more, and, for example, 10 bases or less. The antisense nucleic acid is not limited to a gapmer, and can be a headmer, a tailmer, a mixmer, a blockmer, a totalmer etc. (see, for example, US Patent Application Publication No. 2012/322851A).

A DNA molecule for use as an antisense nucleic acid is preferably an oligodeoxyribonucleotide derived from a region containing the translation initiation site of mRNA encoding the filamin-A gene (e.g., the region at −10 to +10).

Although the antisense nucleic acid and the target nucleic acid preferably have stringent complementarity, some mismatches may be present. The hybridization ability of the antisense nucleic acid to the target nucleic acid generally depends on both the degree of complementarity of the nucleic acids and the length of the antisense nucleic acid. Typically, the longer the antisense nucleic acid for use, the more stable the double strand (or triple strand) that can be formed with the target nucleic acid, irrespective of many mismatches. Those skilled in the art would be able to examine the degree of acceptance of mismatches by using standard methods.

The antisense nucleic acid may be DNA, RNA, chimeric mixtures thereof, or derivatives or modified forms thereof. The antisense nucleic acid may be single-stranded or double-stranded. The stability, hybridization ability, and other properties of the antisense nucleic acid can be improved by modifying the base moiety, sugar moiety, or phosphoric acid skeleton moiety. Additionally, a substance that promotes cell membrane transport (see, for example, Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810, published on Dec. 15, 1988) or a substance that enhances affinity for a specific cell (e.g., a ligand) may be added to the antisense nucleic acid.

The antisense nucleic acid can be synthesized by an ordinary method, such as by using a commercially available automated DNA synthesizer (e.g., Applied Biosystems). For the preparation of modified forms or derivatives of nucleic acids, for example, Stein et al. (1988), Nucl. Acids Res. 16:3209, or Sarin et al., (1988), Proc. Natl. Acad. Sci. U.S.A. 85:7448-7451, can be referred to.

To enhance the action of the antisense nucleic acid in the target cell, potent promoters such as pol II or pol III can be used. Specifically, introducing a construct containing an antisense nucleic acid arranged under control of such a promoter into a target cell ensures the transcription of a sufficient amount of the antisense nucleic acid due to the action of the promoter.

The expression of the antisense nucleic acid can be caused by any promoter (an inducible promoter or a constitutive promoter) known to function in mammalian cells (preferably human cells). For example, a promoter such as the SV40 initial promoter region (Bernoist and Chambon, 1981, Nature 290:304-310), a promoter derived from the 3′-terminal region of the Rous sarcoma virus (Yamamoto et al., 1980, Cell 22:787-797), or the herpes thymidine kinase promoter (Wagner et al., 1981, Proc. Natl. Acad. Sci. U.S.A. 78:1441-1445) can be used.

In an embodiment of the present invention, inhibition of expression by a ribozyme is used (the case of the compound (e)). Although target mRNA may be destroyed by using a ribozyme that cleaves mRNA with a site-specific recognition sequence, a hammerhead ribozyme is preferably used. For the methods for constructing hammerhead ribozymes, for example, Haseloff and Gerlach, 1988, Nature, 334:585-591, can be referred to.

As with the use of the antisense method, a ribozyme may be constructed by using a modified oligonucleotide, for example, with the aim of improving its stability or targeting ability. In order to form an effective amount of a ribozyme in a target cell, it is preferable to use a nucleic acid construct containing DNA encoding the ribozyme, for example, under control of a potent promoter (e.g., pol II or pol III).

Without wishing to be bound by any particular theory, it is speculated from the results of the Test Examples described later that the compound for inhibiting the expression of the filamin-A gene treats PSP by the following mechanism. Filamin-A is an actin-binding protein, and is a molecule that crosslinks F-actin and serves as a cytoskeleton. Because of tau protein having multiple F-actin-binding motifs in its microtubule-binding domain (Reference Literature 34 and 35), tau protein is thought to be abnormally stabilized via F-actin when filamin-A is abundant in quantity or enhanced in its functionality. Thus, when filamin-A is quantitatively high or has enhanced functionality (i.e., pathology of PSP), PSP is assumed to be treatable by reducing the expression levels or functionality of filamin-A to normalize tau protein.

The medical drug of the present invention may contain a compound for inhibiting the expression of the filamin-A gene as a single active ingredient. The medical drug of the present invention may contain only one compound for inhibiting the expression of the filamin-A gene, or two or more compounds for inhibiting the expression of the filamin-A gene. The medical drug of the present invention may contain other active ingredients, and may be administered in combination with other medical drugs (simultaneously, sequentially, or alternately). Examples of other active ingredients or medical drugs include medicaments for Parkinson's disease, such as levodopa and amantadine; and medicaments for depression, such as amitriptyline and tandospirone. Other active ingredients or medical drugs can be used singly or in a combination of two or more.

The medical drug of the present invention can be formulated in accordance with an ordinary method. In formulation, other ingredients acceptable for formulation may be added (e.g., carriers, excipients, disintegrants, buffers, emulsifiers, suspension agents, soothing agents, stabilizers, preservatives, antiseptics, and physiological saline).

Examples of carriers include, but are not limited to, cationic liposomes, such as Lipofectin (trademark), Lipofectamine 2000 (trademark), and Oligofectamine (trademark); and cationic polymers, such as poly(L-lysine), DEAE-dextran, polyethylenimine, and chitosan.

The excipient for use can be lactose, starch, sorbitol, D-mannitol, sucrose, etc. The disintegrant for use can be starch, carboxymethyl cellulose, calcium carbonate, etc. The buffer for use can be phosphate, citrate, acetate, etc. The emulsifier for use can be gum arabic, sodium alginate, tragacanth, etc. The suspension agent for use can be glyceryl monostearate, aluminum monostearate, methyl cellulose, carboxymethyl cellulose, hydroxymethy cellulose, lauryl sodium sulfate, etc. The soothing agent for use can be benzyl alcohol, chlorobutanol, sorbitol, etc. The stabilizer for use can be propylene glycol, diethyl sulfite, ascorbic acid, etc. The preservative for use can be phenol, benzalkonium chloride, benzyl alcohol, chlorobutanol, methylparaben, etc. The antiseptic for use can be benzalkonium chloride, p-hydroxybenzoic acid, chlorobutanol, etc.

The dosage form for formulation is not particularly limited. Examples of dosage forms include tablets, powdered drugs, subtle granules, granules, capsules, syrup, injectable drugs, and inhalants. The medical drug of the present invention contains the compound for inhibiting the expression of the filamin-A gene (or an active ingredient) in an amount necessary for achieving an expected therapeutic effect (or a preventive effect) (i.e., a therapeutically effective amount). Although the content of the compound (or the amount of an active ingredient) in the medical drug of the present invention generally varies according to the dosage form, the content of the compound is set within the range of, for example, about 0.01 wt % to about 95 wt % so as to achieve a desired dose. The medical drug of the present invention is administered to a subject perorally or parenterally (e.g., intravenous, intraarterial, subcutaneous, intradermal, intramuscular, or intraperitoneal injection, transdermal, transnasal, transmucosal, intracerebral, or intrathecal administration) according to the dosage form. Examples of intracerebral administration include administration through a catheter, intracerebral implant of the formulation in its sustained-release form, and introduction of the formulation into intracerebral cells by electroporation. These routes of administration are not mutually exclusive, and two or more of them may be freely selected and used in combination (e.g., performing intravenous injection at the same time as peroral administration or after a predetermined time from peroral administration). The compound for inhibiting the expression of the filamin-A gene in the form of a nucleic acid construct (e.g., an embodiment in which RNAi is used) can be administered ex vivo, as well as in vivo.

Although the “subject” to whom the medical drug of the present invention is administered is typically a human, the medical drug is also expected to be applied to mammals other than humans (including pet animals, domestic animals, and laboratory animals; specifically, animals such as mice, rats, guinea pigs, hamsters, monkeys, cows, pigs, goats, sheep, dogs, and cats). The subject may be, for example, a subject with an increased expression level of filamin-A and/or 4-repeat tau in neurons and/or glial cells, or a subject with an expression level of 4-repeat tau higher than the expression level of 3-repeat tau in neurons and/or glial cells. Although the dose varies, for example, according to the symptoms, age, gender, and body weight of the subject (e.g., a patient), those skilled in the art would be able to determine an appropriate dosage. In setting a dosing schedule, the symptoms of the subject (e.g., a patient) and the duration of the drug's effect can be taken into consideration.

As is clear from the above description, the present application also provides a method for treating PSP, characterized by the administration of a therapeutically effective amount of the medical drug of the present invention to a patient with PSP. The phrase “method for treating PSP” as used here, as with “medicament,” is intended to include both therapeutic and prophylactic methods, and includes methods to prevent or delay the progress of symptoms. The present application also provides a 4-repeat tau expression inhibitor containing a compound for inhibiting the expression of the filamin-A gene, a phosphorylated 4-repeat tau inhibitor containing a compound for inhibiting the expression of the filamin-A gene, and a 4-repeat tau aggregation inhibitor containing a compound for inhibiting the expression of the filamin-A gene. The phosphorylated 4-repeat tau inhibitor contains an agent for reducing the amount of or concentration of phosphorylated 4-repeat tau in the cells expressing filamin-A. The components, their content, administration forms, and the like of such an agent are as described for the “medicament” above.

### 2. Search for Compounds Effective in the Treatment of Progressive Supranuclear Palsy (PSP) (Assessment and Screening)

Another aspect of the present invention relates to the search for compounds effective in the treatment of PSP (assessment and screening). The inventors' study identified filamin-A as a gene responsible for PSP and also revealed that the increased expression of filamin-A resulted in increased 4-repeat tau and increased phosphorylated tau. In this aspect, “increased expression of filamin-A” includes an increase in mRNA encoding filamin-A, and “phosphorylated tau” includes phosphorylated 4-repeat tau. On the basis of these findings, the present invention provides a method for assessing the efficacy of a test substance on PSP (“the assessment method of the present invention” below) that uses increased expression of filamin-A, increased 4-repeat tau associated with an increased expression level of filamin-A, or increased phosphorylated tau associated with an increased expression level of filamin-A as an indicator. The assessment method of the present invention is useful in searching for the candidates for medicaments for PSP (i.e., screening).

The assessment method of the present invention includes the following steps (i) and (ii):

(i) the step of brining a test substance into contact with a cell expressing filamin-A; and
(ii) the step of detecting the expression level of filamin-A, the amount of 4-repeat tau, and/or the amount of phosphorylated tau in the cell above to determine the efficacy of the test substance on the basis of the detection results, wherein a decreased expression level of filamin-A (indicator 1), a decreased amount of 4-repeat tau (indicator 2), and/or a decreased amount of phosphorylated tau (indicator 3) is the indicator for the efficacy of the test substance.

In step (i), cells expressing filamin-A are prepared. The cells expressing filamin-A can be of any type, and can be cells in vivo, cells taken from a living organism, or cultured cells. The cells expressing filamin-A. are, for example, preferably cells that highly express filamin-A. In an embodiment of the present invention, the cells expressing filamin-A. are cells, preferably cell lines, and more preferably lymphocyte cell lines derived from a patient with PSP. The cells are preferably immortalized cells. For example, lymphocytes of a patient with PSP can be harvested and immortalized to prepare lymphocyte cell lines, and the lymphocyte cell lines can be used in the assessment method of the present invention. In a preferable embodiment, immortalization is performed by collecting peripheral blood from a patient with PSP, and infecting B lymphocytes with Epstein-Barr virus. Alternatively, the assessment method of the present invention can use PSP patient-derived induced pluripotent stem (iPS) cells (iPS cells prepared by using cells harvested from a patient), or such iPS cells differentiated into neurons, glial cells, or brain organoids (PSP patient-derived neurons, glial cells, brain organoids). Additionally, cells for use in the assessment method of the present invention can also be prepared by genetic modification using, for example, gene targeting or genome editing techniques (e.g., ZFN, TALEN, and CRISPER/Cas9). Examples of cells for use in genetic modification include fibroblasts, cardiomyocytes, smooth myocytes, adipocytes, osteocytes, chondrocytes, osteoclasts, parenchymal cells, epidermal keratinocytes (keratinocytes), epithelial cells (e.g., cutaneous epithelial cells, corneal epithelial cells, conjunctival epithelial cells, oral mucosal epithelia, hair follicle epithelial cells, oral mucosal epithelial cells, airway mucosal epithelial cells, and intestinal mucosal epithelial cells), endothelial cells (e.g., corneal endothelial cells, and vascular endothelial cells), neurons, glial cells, splenocytes, pancreatic β cells, mesangial cells, Langerhans cells, hepatocytes, myeloid cells, hematocytes (leukocytes), precursor cells thereof, stem cells thereof, and cell lines (e.g., HeLa cells, CHO cells, Vero cells, HEK293 cells, HepG2 cells, COS-7 cells, NIH3T3 cells, and Sf9 cells). Although human cells are preferably used, the use of cells from other animal species (e.g., monkeys, cows, horses, rabbits, mice, rats, guinea pigs, and hamsters) is not excluded. PSP patient-derived immortalized lymphocytes with an increased expression level of filamin-A are not only useful for the assessment method of the present invention, but also have a great value in themselves as the cells are usable in research on PSP, or development of medicaments for PSP. In an embodiment of the present invention, the cells expressing filamin-A are cells derived from a transgenic animal (e.g., a transgenic mouse) having the filamin-A gene (e.g., human filamin-A gene) introduced, and are preferably cell lines. The cells are preferably those prepared by isolating primary neural stem cells or primary glial cells from the fetal brain tissue of a transgenic animal and immortalizing them. Immortalization can be performed by introducing to the cells, for example, SV40 T-antigen gene, HPV E6E7 gene, v-abl gene, myc gene, human telomerase reverse transcriptase (hTERT) gene, or a combination of these genes. The virus for use as a vector for introducing the gene can be any virus, and is preferably lentivirus, adenovirus, or retrovirus. The cells derived from a transgenic animal are preferably embryonic fibroblasts (MEF) obtained from fetal tissue mass of a transgenic animal. These cells can be used as stable cell lines due to their high proliferative capacity.

The “contact” in step (i) is typically performed by administering a test substance in vivo when the cells are present in a living organism. When the cells are those harvested from a living organism or cultured cells, the contact is typically performed by adding a test substance to a culture broth (medium) during culture. The time when a test substance is added is not particularly limited. Thus, after the start of culturing cells in a medium free of a test substance, the test substance may be added at a given point in time. Alternatively, cell culture can be started in a medium containing a test substance. Culture conditions can be standard conditions for cells for use.

The test substance for use can be organic or inorganic compounds of different molecular sizes. Examples of organic compounds include nucleic acids, peptides, proteins, lipids (simple lipids, complex lipids (e.g., phosphoglycerides, sphingolipids, glycosyl glycerides, and cerebrosides)), prostaglandins, isoprenoids, terpenes, steroids, polyphenols, catechins, and vitamins (e.g., B1, B2, B3, B5, B6, B7, B9, B12, C, A, D, and E). Existing or candidate ingredients for medical drugs or nutritional foods are also preferable test substances. Plant extracts, cell extracts, and culture supernatants may be used as a test substance. Existing medicinal agents (e.g., the library of drugs approved by the United States Food and Drug Administration (FDA)) can also be used as a test substance.

Various compound libraries (e.g., Ligand Box) are available (e.g., available from Asinex or Namiki Shoji Co., Ltd.), and these compound libraries can also be used. The test substance may be derived from natural products or synthesized. In the latter case, for example, combinatorial synthesis techniques can be used to construct an efficient screening system. Additionally, two or more test substances may be added at the same time to investigate the interaction and synergistic action between the test substances.

The time period for the contact of the test substance can be freely set. For example, if the cells are those harvested from a living organism or cultured cells, the contact time period is, for example, 10 minutes to 1 week, and preferably 1 hour to 3 days. The contact may be divided and performed multiple times.

In step (ii) following step (i), the expression of filamin-A (if the expression of filamin-A is an indicator), the amount of 4-repeat tau (if the amount of 4-repeat tau is an indicator), and/or the amount of phosphorylated tau (if the amount of phosphorylated tau is an indicator) in the cells that have come into contact with a test substance is detected, and the efficacy of the test substance is determined based on the detection results. Specifically, the present invention uses the following three indicators.

Indicator 1: decreased expression of filamin-A
Indicator 2: a decreased amount of 4-repeat tau
Indicator 3: a decreased amount of phosphorylated tau

These indicators 1 to 3 are not mutually exclusive, and two or three indicators can be used in combination. A combination of two or three indicators provides more informative determination results. Thus, preferably, two or three indicators are used in combination; more preferably, indicators 1 to 3 are all used in combination.

With the use of indicator 1, the test substance is determined to be effective when the expression of filamin-A is decreased. With the use of indicator 2, the test substance is determined to be effective when the amount of 4-repeat tau is decreased. In the same manner, with the use of indicator 3, the test substance is determined to be effective when the amount of phosphorylated tau is decreased. The potency (level) of the action and effect of the test substance may be determined based on the level of the decrease in expression of filamin-A (with indicator 1), the level of the decrease in the amount of 4-repeat tau (with indicator 2), or the level of the decrease in the amount of phosphorylated tau (with indicator 3). When multiple test substances are used, the potency of the action and effect of each test substance may be compared and assessed based on the level of the decrease in the expression of filamin-A (with indicator 1), the level of the decrease in the amount of 4-repeat tau (with indicator 2), or the level of the decrease in the amount of phosphorylated tau (with indicator 3).

The expression of filamin-A can be detected, for example, by real-time quantitative PCR, microarray, RNA-Seq, immunological assays (e.g., western blotting or ELISA), or proteome analysis using mass spectrometry. 4-repeat tau can be detected, for example, by immunological assays (e.g., western blotting or ELISA) or proteome analysis using mass spectrometry. In the same manner, phosphorylated tau can be detected, for example, by immunological assays (e.g., western blotting or ELISA) or proteome analysis using mass spectrometry.

Typically, cells that are not brought into contact with a test substance (other conditions are the same) are prepared as a control for comparison (“control cells” below), and the expression of filamin-A (indicator 1), the amount of 4-repeat tau (indicator 2), and/or the amount of phosphorylated tau (indicator 3) in the control cells is also detected. Then, the efficacy of the test substance is determined by making a comparison with the detection results of the control cells (preferably, quantitative determination rather than qualitative determination).

Determination of the action or effect of a test substance based on a comparison with the control provides a more reliable determination result.

As mentioned above, the assessment method of the present invention is useful in searching for (i.e., screening for) candidate medicaments for PSP. In other words, the present invention allows for the identification of active ingredient candidates or lead compounds for medicaments. If the assessment method of the present invention is used in screening, an effective test substance is selected based on the determination result of step (ii). If the selected substance has sufficient efficacy, the substance as is can be used as an active ingredient for medicaments for PSP. If the selected substance does not have sufficient efficacy, the substance may be modified (e.g., chemical modification) to increase its efficacy and then used as an active ingredient for medicaments for PSP. Of course, even a substance having sufficient efficacy may be modified in the same manner with the aim of further increasing efficacy.

### 3. Disease Model of Progressive Supranuclear Palsy (PSP)

Another aspect of the present invention relates to a non-human mammal that reproduces the pathology of PSP. The non-human mammal of the present invention is useful as a disease model of PSP (model animal). A typical example of the non-human mammal of the present invention is, but is not limited to, a transgenic animal (“TG animal” below). For example, genetically modified animals prepared by using a genome editing technique (e.g., gene knock-in) or a viral vector (e.g., gene expression induction by an adeno-associated viral vector) also fall under the category of non-human mammals as a disease model for PSP. Transgenic non-human mammals (TG animals) refer to mammals other than humans prepared by introducing exogenous DNA at an early stage of development to allow all cells constituting the animal to possess the exogenous DNA or their offspring (that possesses the exogenous gene).

The model animal of the present invention is not particularly limited in terms of mammalian species (genus), and can be a mouse, a rat, a guinea pig, a hamster, a rabbit, a dog, a cat, a monkey, etc. The model animal is preferably a rodent such as a mouse or a rat, and most preferably a mouse.

Typically, exogenous DNA in the present invention includes the human filamin-A gene (e.g., the human filamin-A gene having the sequence represented by SEQ ID NO: 2 or SEQ ID NO: 4) as a transgene. A homolog, orthologue, or mutant of the human filamin-A gene may be used as a transgene as long as its forced expression results in an increase in the amount of 4-repeat tau or an increase in the amount of phosphorylated tau via an increase in the expression of filamin-A. As used here, the term “mutant” refers to a sequence that is identical or homologous to a portion of the sequence of the human filamin-A gene, but has differences in comparison between its entire sequence and the sequence of the human filamin-A gene. An example of mutants of the human filamin-A gene is a DNA sequence containing one or multiple base substitutions, deletions, insertions, and/or additions in the DNA sequence of the human filamin-A gene.

Mutants may be those naturally occurring or artificially constructed by using genetic engineering techniques. The number of copies of the transgene is not limited to any particular number, and is, for example, 1 to 100.

The exogenous DNA preferably contains an enhancer for activating the transcription of the transgene. The term “enhancer” refers to a sequence that directly or indirectly acts on a promoter to enhance the transcriptional activity. The enhancer generally acts on a promoter from a distance. The location of the enhancer within exogenous DNA may be upstream or downstream of the promoter. The enhancer is not particularly limited as long as the enhancer can act on the promoter used in exogenous DNA to increase its transcriptional activity.

The non-human mammal of the present invention (typical example: TG animal) contains the above exogenous gene in heterozygous or homozygous form. In other words, the genotype of the above exogenous gene is a heterozygote or homozygote.

Methods for creating TG animals, which are a typical example of the non-human mammal of the present invention, include microinjection that directly injects DNA into the pronucleus of a fertilized egg, methods using a retroviral vector, and methods using ES cells. The following describes microinjection using mice as a specific example of methods for creating TG animals of the present invention.

In microinjection, a fertilized egg is first collected from the fallopian tube of a female mouse confirmed to have mated, and is cultured, followed by injecting a desired DNA construct (exogenous DNA) into the pronucleus. The form of the DNA construct is not particularly limited, but is preferably linear or cyclic from the standpoint of introduction efficiency. Particularly preferably, a linearly prepared DNA construct is used. The DNA construct is prepared such that the gene of interest is efficiently incorporated into the chromosome and its good expression is ensured. The DNA construct contains a transgene (typically, human filamin-A gene) and a promoter (optionally including an enhancer sequence, a selectable marker, an origin of replication, a terminator sequence, etc. as necessary).

The fertilized egg that has completed the injection operation is implanted into the fallopian tube of a pseudopregnant mouse, and the implanted mouse is reared for a predetermined period of time to obtain baby mice (F0). To confirm whether the transgene is properly incorporated into the chromosomes of a baby mouse, DNA is extracted from the tail of the baby mouse and subjected to Southern hybridization analysis, slot blot (dot blot) analysis, PCR analysis, etc.

An identified transgenic mouse is then mated with a wild-type mouse to obtain a heterozygous transgenic mouse (carrying exogenous DNA in a heterozygous form). A homozygous transgenic mouse (carrying exogenous DNA in a homozygous form) can be obtained by mating thus-obtained male and female heterozygous transgenic mice. For breeding or maintenance, the male and female homozygous transgenic mice can be mated.

The non-human mammal of the present invention (typical example: TG animal) reproduces the pathology of PSP. Typically, the non-human mammal of the present invention shows a phenotype of increased 4-repeat tau and/or increased phosphorylated tau in neurons or glial cells. Because of this feature, the non-human mammal of the present invention is useful in the search for medicaments for PSP and the verification of their efficacy. For example, substances that improve (including cure) the characteristic phenotype (pathology) exhibited by the non-human mammal can be identified as medicament candidates for PSP. For example, substances that improve (including cure) phenotypes characteristic of the non-human mammal of the present invention (pathology) can be identified as medicament candidates for PSP. Additionally, an increase in 4-repeat tau and/or an increase in phosphorylated tau occurs in the non-human mammal of the present invention as the basis of phenotypes (pathology). Thus, effectiveness of a test compound can be determined by detecting the amount of 4-repeat tau or the amount of phosphorylated tau (e.g., detection by fluorescence biological imaging, western blotting, immunostaining etc.) and using the change in the amount as an indicator.

### 4. Biomarker for Progressive Supranuclear Palsy (PSP)

The biomarker for progressive supranuclear palsy (PSP) of the present invention contains filamin-A. For example, if the expression level of filamin-A in neurons and/or glial cells derived from a subject is greater than the expression level of filamin-A in neurons and/or glial cells derived from a healthy subject (e.g., more than twofold or threefold), the subject can be determined to have PSP.

### EXAMPLES

The following study was conducted to create a novel therapeutic strategy for progressive supranuclear palsy (PSP).

### 1. Method

### (1) Analysis Target

A Japanese family including identical twins (Twin-A, Twin-B) who developed PSP at the same time and 32 cases among the registered cases in the brain bank of the Institute for Medical Science of Aging at Aichi Medical University were analysis targets. The 32 cases included 9 cases of PSP (PSP-1 to PSP-9), 3 cases of corticobasal degeneration (CBD) (CBD-1 to CBD-3), 3 cases of AD (AD-1 to AD-3), 4 cases of Parkinson's disease (PD) (PD-1 to PD-4), 3 cases of dementia with Lewy bodies (DLB) (DLB-1 to DLB-3), 5 cases of amyotrophic lateral sclerosis (ALS) (ALS-1 to ALS-5), and 5 cases of healthy control (Normal-1 to Normal-5). The pathological diagnosis was made based on the diagnostic criteria for each disease (Reference Literature 7 to 12). Age-related changes were assessed using Braak NFT staging (grade 0, I-VI) (Reference Literature 13), AT8 staging (grade 0, I-VI) (Reference Literature 14), argyrophilic grain (AG) staging (grade 0, I-III) (Reference Literature 15), and a score of the Consortium to Establish a Registry for Alzheimer's Disease (CERAD) (grade 0, A-C) (Reference Literature 16). For analysis, informed consent was obtained in writing from the subjects or their relatives. The research plan for this study was reviewed and approved by the ethics committees of Nagoya University, Aichi Medical University, and Yokohama City University.

### (2) Microsatellite Marker

To confirm the match of genomic DNA between Twin-A and Twin-B, the genotype of 12 microsatellite markers was analyzed using fluorescent primers of the ABI PRISM Linkage Mapping Set version 2.5 (Applied Biosystems), and the relationship between the identical twins was evaluated.

### (3) Human Neuropathological Analysis

An autopsy brain tissue was immobilized by 20% formalin. A brain specimen was embedded in paraffin, and a section was prepared with a thickness of 4.5 μm. The section was subjected to hematoxylin and eosin (H&E) staining, Kluver-Barrera (KB) staining, and Gallyas-Braak staining. The primary antibodies for use were 3R-tau antibody (RD3), 4R-tau antibody (RD4), and phosphorylated tau antibody (AT8). For staining, an Envision Kit (DAB) (Wako) was used.

### (4) Immortalized Lymphocytes

Peripheral blood was collected from Twin-A, Twin-B, and three siblings (II-1, 11-2, and 11-3), and B lymphocytes were infected with Epstein-Barr virus and immortalized. These immortalized lymphocytes were cultured in an RPMI 1640 medium (Gibco) supplemented with 10% fetal bovine serum (FBS) at 37° C. in 5% CO₂.

### (5) Sanger Sequencing of MAPT Gene

Sanger sequencing was performed on exon 10 and nearby introns of the MAPT gene using a 3730xl DNA analyzer (Applied Biosystems). Polymerase chain reaction (PCR) was performed using a Multiplex PCR Assay Kit (Takara). The primers for use were the following: forward 5′-GGATGTGACTCAACCTCCCG-3′ (SEQ ID NO: 5) and reverse 5′-CGGGCTACATTCACCCAGAG-3′ (SEQ ID NO: 6).

### (6) Whole Exome Analysis

The genomic DNA was extracted from peripheral blood of Twin-A, and whole exome analysis was performed using a SureSelect Human All Exon V6 kit (Agilent Technologies). The captured libraries were sequenced using a HiSeq 2500 system (Illumina). Reads were aligned in accordance with the human reference sequence GRCh37 by Novoalign, and duplicate reads were removed using Picard. Variant calling was performed using a Genome Analysis Toolkit (GATK) and annotated using ANNOVAR. The average coverage depth was 69.7×, with more than 20 reads covering 95.1° of the coding region.

### (7) Analysis of the Number of Copies

The number of copies was calculated from the whole exome analysis data using eXome Hidden Markov Model v1.0 (XHMM) (Reference Literature 17 and 18). First, BAM files of Twin-A and 513 healthy Japanese males were created. The average depth of each target region was then calculated from the BAM files for each sample by using GATK DepthOfCoverage and integrated into a samples-by-target matrix. Targets showing outliers in size, depth, and GC content, and samples showing outliers in the average value or standard deviation of depth, were excluded. The depth of the integrated matrix was centered according to the average value for each target and used in principal component analysis. The Z-score of each target was calculated for each sample, and the number of copies was calculated from the Z-score using the XHMM algorithm. Z-scores were visualized using SignalMap Version 1.9.0.05 (Roche Nimblegen). The abnormal regions in the number of copies detected by XHMM were re-evaluated by microarray and real-time quantitative PCR, described below.

### (8) Chromosome Microarray

As stated above, a high-resolution chromosome microarray was performed on the genomic DNA extracted from Twin-A, Twin-B, and their three siblings (II-1, 11-2, and 11-3) by using CytoScan HD Array (Affymetrix). Data analysis was performed with Chromosome Analysis Suite software v1.2.0.225 (Affymetrix). 250 ng of the genomic DNA purified by ethanol precipitation was treated with restriction enzyme Nsp1 and ligated with an adaptor using T4 DNA ligase. PCR amplification was then performed using primers targeting the adaptor sequence and Titanium Taq DNA Polymerase (Affymetrix). The PCR product was purified using ceramic beads, fragmented with DNase I, and biotin-labeled with terminal deoxynucleotidyl transferase. The labeled DNA was hybridized with a CytoScan HD Chip using Gene Chip Hybridization Oven 640 (Affymetrix). The chip was washed and then scanned on a GeneChip Fluidics Station 450 (Affymetrix).

### (9) RNA Reverse Transcription

RNA was extracted from cells using an miRNeasy Mini Kit (Qiagen), and complementary DNA (cDNA) was prepared from 1.0 μg of RNA using an ImProm-II™ Reverse Transcription System (Promega).

### (10) Real-Time Quantitative PCR

A Thunderbird SYBR qPCR Mix (Toyobo) and a CFX96 system (BioRad) were used. Of the primers of the genomic DNA, the sequences of MECP2, TKTL1, G6PD, and the intergenic region between CTAG1B and CTAG2, and GAB3 for use were the same as those previously reported (Reference Literature 19). FLNA was newly designed, and the sequence is the following: forward 5′-AAGGGGGAGTACACACTGGT-3′ (SEQ ID NO: 7) and reverse 5′-CACCACAACGCGGTAGGG-3′ (SEQ ID NO: 8). Of the cDNA primers, FLNA, RPL10, ATP6AP1, GDI1, and GUSB for use were the same sequences as those previously reported (reference literature 19). PCR was performed under the following conditions: 95° C. for 3 minutes, followed by 40 cycles of 95° C. for 10 seconds and 55° C. for 30 seconds. Relative gene expression levels were calculated by the 2-ΔΔCt method, determining a housekeeping gene as a reference and a control sample as a calibrator for each experiment.

### (11) X Chromosome Inactivation Analysis

The genomic DNA was treated with methylation-sensitive restriction enzymes HpaII and HhaI (Takara), and the CAG repeat region of the FRAXA gene was amplified using primers with fluorescent probes (Reference Literature 20). The PCR product was subjected to fragment analysis with an ABI PRISM 3500 Genetic Analyzer (Applied Biosystems). An X chromosome inactivation ratio of less than 80:20 was considered a random pattern, a ratio of 80:20 or greater was considered a skewed pattern, and a ratio of 90:10 or greater was considered a markedly skewed pattern (Reference Literature 20). Data processing was performed using Peak Scanner software 2 (Applied Biosystems).

### (12) DNA Plasmid

Because mCherry-Filamin-A-N-9 (Addgene, plasmid 55047) has a mutation at base 7876 of the FLNA cDNA sequence, it was substituted with the wild-type FLNA sequence using a KOD Plus Mutagenesis Kit (Toyobo), and used as a mCherry-FLNA vector. To eliminate the actin-binding ability, a mCherry-FLNA^{Ala39Gly} vector with a mutation at base 116 of the FLNA cDNA sequence was prepared according to a previous report (Reference Literature 32). Additionally, the entire FLNA cDNA sequence in the mCherry-FLNA vector was deleted, and the result was used in negative control as a mCherry-empty vector. The cDNA sequences of various sub-cloned genes were inserted into the mCherry-empty vector using In-Fusion HD (Takara), and a mCherry-RPL10 vector, a mCherry-GDI1 vector, a mCherry-FAM3A vector, and a mCherry-G6PD vector were prepared. GFP-tagged human 0N4R tau sequences were inserted into a pDEST 12.2 vector and a pLenti CMV neo-vector, and GFP-tagged 4R-tau (GFP-4R-tau) was overexpressed in cultured cells (Reference Literature 5).

### (13) Transfection

HEK293 cells were cultured in a DMEM medium (Nakarai Tesque) supplemented with 10% FBS at 37° C. in 5% CO₂. Lipofectamine 2000 (Invitrogen) was used for the transfection of HEK293 cells with DNA plasmids, and Lipofectamine 3000 (Invitrogen) was used for primary astroglia. The cells were collected 48 hours after transfection and used in each analysis.

### (14) Small Interfering RNA

Small interfering RNA (siRNA) was purchased from Invitrogen. ID numbers are as follows: FLNA siRNA #1 (s5275, the sequence of sense RNA: SEQ ID NO: 9, the sequence of antisense RNA: SEQ ID NO: 10), FLNA siRNA #2 (s5276, the sequence of sense RNA: SEQ ID NO: 11, the sequence of antisense RNA: SEQ ID NO: 12), FLNA siRNA #3 (s5276, the sequence of sense RNA: SEQ ID NO: 13, the sequence of antisense RNA: SEQ ID NO: 14), and control siRNA (Silencer Negative Control siRNA No. 1, 4390843). Electroporation (Neon, Invitrogen) was performed to transfect immortalized lymphocytes with each siRNA. The transfection conditions were 1 pulse of 30 ms, and 1350 V; the cells were collected after 24 hours and used in each analysis.

### (15) Cycloheximide Chase Experiment

A cycloheximide chase experiment was performed to investigate the stability of tau protein in cultured cell experiments (Reference Literature 21). GFP-4R-tau and mCherry-FLNA were expressed in HEK293 cells, and cycloheximide (100 μg/ml) was added to inhibit new protein synthesis. The cells were collected at each observation time point and used in western blotting. The expression level of GFP-4R-tau was normalized with the expression level of GAPDH at the start of addition.

### (16) Sarkosyl-Insoluble Tau

Sarcosyl-insoluble tau was collected from GFP-4R-tau-expressing HEK293 cells and a human autopsy brain (Reference Literature 22). Each sample was dissolved in a 10-fold volume TBS buffer [50 mM Tris/HCl (pH of 8.0), 274 mM NaCl, 5 mM KCl, protease inhibitor cocktail (04693159001 Roche), phosphatase inhibitor (04906837001 Roche)] and made into a homogenate (Ho). Ho was ultracentrifuged (27,000×g, 4° C., 20 minutes), and the supernatant was used as a TBS-soluble fraction (S1). The precipitate was dissolved in a high salt/sucrose buffer [0.8 M NaCl, 10% sucrose, 10 mM Tris/HCl (pH of 7.4), 1 mM EDTA, protease inhibitor cocktail, phosphatase inhibitor], and ultracentrifuged under the same conditions as above. The supernatant was dissolved in sarkosyl (final concentration: 1%), and kept warm at 37° C. for 1 hour, followed by ultracentrifugation (150,000×g, 4° C., 1 hour). The precipitate was dissolved in a TE buffer [10 mM Tris/HCl (pH of 8.0), 1 mM EDTA] to make sarkosyl-insoluble fraction P3.

### (17) Coimmunoprecipitation

HEK293 cells on a 100-mm plate were transfected with 8 μg of a vector for expressing GFP-4R-tau or 8 μg of a vector for expressing mCherry-FLNA. The cells were harvested with trypsin-EDTA (Gibco) after 48 hours, washed 4 times with PBS, and then dissolved in Cell Lysis Buffer M (Wako) [20 mM Tris-HCl (pH of 7.4), 200 mM NaCl, 0.05% Nonidet P-40, 2.5 mM MgCl2]. Immunoprecipitation was performed using 5 μg of a tau antibody (TAU-5, ab80579, Abcam) and a Dynabeads Protein G Immunoprecipitation Kit (Invitrogen), and the precipitates were used in various western blotting assays.

### (18) Lentivirus

HEK293T cells were transfected with a packaging vector and a lentiviral vector using Lipofectamine 2000 (Invitrogen), thereby producing lentiviral particles (Reference Literature 5). After 48 hours from the transfection, the lentivirus-containing supernatant was collected and frozen at −80° C. for storage.

### (19) Rat Primary Astroglia

The cerebral cortex of a one-day-old Wistar rat was harvested and kept warm at 37° C. for 15 minutes using a Hanks' balanced salt solution (HBSS) supplemented with 0.25% trypsin and DNase I (Reference Literature 23). Mixed glia was cultured in a DMEM medium supplemented with 20% FBS in a T75 flask, and the medium was replaced every 3 days. As soon as the cells became confluent, the cells were shaken at 37° C. and at 200 rpm for 24 hours with a constant-temperature shaker to remove microglia and oligodendrocytes. The animal experiments complied with the National Institutes of Health Guide for the Care and Use of Laboratory Animals and were approved by the animal testing committee of Nagoya University.

### (20) Western Blotting

Western blotting was performed as previously reported (Reference Literature 24 and 25). The primary antibodies are as follows: FLNA antibody (Santa Cruz Biotechnology (SCB), sc-17749, sc-28284), EMD antibody (SCB, sc-25284), RPL10 antibody (Abcam, ab138978), DNASE1L1 antibody (SCB, sc-134320), TAZ antibody (SCB, sc-293183), ATP6AP1 antibody (Abnova, H00000537-M01), GDI1 antibody (GeneTex, GTX54148), FAM50A antibody (SCB, sc-100967), PLXNA3 antibody (SCB, sc-374662), LAGE3 antibody (SCB, sc-515776), UBL4A antibody (Proteintech, 14253-1-AP), SLC10A3 antibody (Novus Biologicals, NBP1-79316), FAM3A antibody (R&D, MAB2865-SP), G6PD antibody (SCB, sc-373886), IKBKG antibody (SCB, sc-8032), CTAG1B antibody (SCB, sc-53869), GAPDH antibody (Abcam, ab8245), mCherry antibody (Abcam, ab167453), GFP antibody (MBL, 598), Tau-5 antibody (Abcam, ab80579), RD4 antibody (Millipore, 05-804), AT8 antibody (Invitrogen, MN1020), PHF-1 antibody (provided by Dr. Peter Davie), HSP90 antibody (Cell Signaling Technology (CST), 4874), HSP70 antibody (CST, 4872), HSP40 antibody (CST, 4871), and Ubiquitin antibody (CST, 3933). A LAS3000 imaging system (Fujifilm) was used for image capturing. Signals were quantified with Image Gauge software version 4.22 (Fujifilm) and used in comparison of protein expression levels.

### (21) Fluorescent Immunostaining

For astroglial cell staining, the cells were immobilized with 4% paraformaldehyde (PFA) for 30 minutes and permeabilized with 1% Triton X-100 (Sigma) for 5 minutes. The cells were then blocked with a Tris-NaCl-blocking (TNB) buffer (PerkinElmer). For human autopsy brain tissue staining, formalin-immobilized paraffin-embedded sections were deparaffinized and treated with microwave heat using a 50 mM citrate buffer (pH of 6.0) for 15 minutes, followed by blocking with a TNB buffer. The primary antibodies for use were the following: mCherry antibody (Abcam, ab167453), AT8 antibody (Invitrogen, MN1020), FLNA antibody (SCB, sc-28284), and GFAP antibody (Abcam, ab4674). The secondary antibodies for use were those of Alexa Fluor series (Invitrogen). The cells were encapsulated with a ProLong gold antifade reagent (Invitrogen, P36930). The images were captured with a confocal laser microscope (LSM710, Carl Zeiss).

### (22) Statistical Analysis

Analysis was performed using R software (ver. 3.5.1). The Student's t test was used in comparison between two groups. Parametric multiple group comparison was performed with the Tukey-Kramer test, and non-parametric multiple group comparison was performed with the Steel-Dwass test. The correlations were evaluated by the test of no correlation of the Pearson product-moment correlation coefficient. Values were expressed as median±standard error of the mean (SEM). The P value was significant when it was less than 0.05, and expressed in the figures as the following: ***P<0.001, **P<0.01, and *P<0.05.

### (23) Transgenic Mouse

A transgene (SEQ ID NO: 15) was formed of a full-length human FLNA cDNA sequence and a FLAG tag sequence located at the 3′terminus of the sequence and designed to be expressed downstream of the CAG promoter. This transgene was injected into fertilized eggs of C57BL/6J strain to prepare transgenic mice.

### 2. Results

Tests below were conducted according to the method described in section 1 above.

### Test Example 1: Identical Twins Who Developed PSP at the Same Time

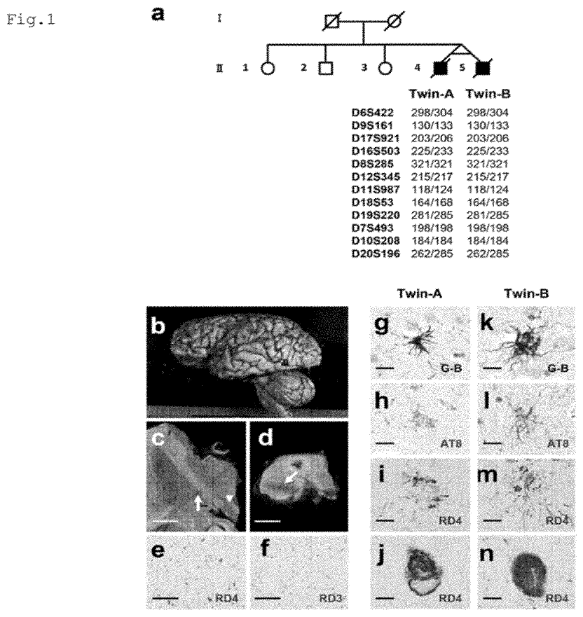

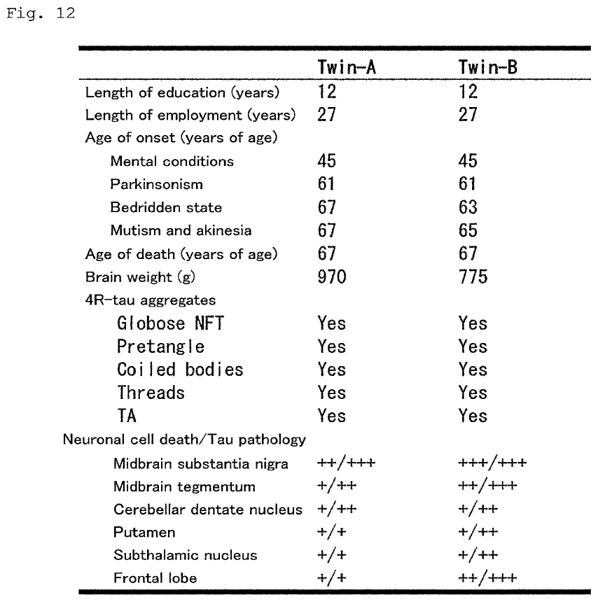

The present inventors first conducted pathological analysis of identical twins who developed PSP at the same time. Sanger sequencing showed no pathological mutations in the MAPT gene, and a microsatellite marker confirmed that they were a monozygotic twin pair (FIG. 1a). Both had been employed after graduation from high school, but started to have depression and showed disinhibited behavior from the age of 45; they took a leave of absence, and met the conditions for clinical diagnosis of frontotemporal dementia behavior variants (Reference Literature 26). Their higher cerebral function progressively declined; at the advanced stage, supranuclear vertical eye movement disorder, muscle rigidity predominant in the trunk, and unsteady gait were observed. Both died of pneumonia at the age of 67. Their three siblings (II-1, II-2, and II-3) were neurologically normal. In pathological anatomy, atrophy of the frontal lobe, globus pallidus, and midbrain was grossly observed (FIGS. 1b to 1d). Microscopic examination revealed neurological deficits, gliosis, and 4R-tau-positive globose-type NFT or TA in the subthalamic nucleus, internal globus pallidus, midbrain tegmentum, and cerebellar dentate nucleus (FIGS. 1g to 1n). Based on the diagnostic criteria, a pathological diagnosis of PSP was made (Reference Literature 7). However, compared with previously reported PSP cases (48 PSP cases: brain weight of 1.1±0.02 kg, Reference Literature 27), Twin-A and Twin-B had a lower brain weight of 970 g and 775 g, respectively. The severity of clinical symptoms and neuropathological findings (FIG. 12) was more advanced in Twin-B than in Twin-A. Twin-B had gray matter heterotopia in the anterior horn of the lateral ventricle and cerebellum (FIG. 5). FIGS. 6 and 7 show additional information on neuropathological findings and neuroradiological images.

### Test Example 2. Identification of FLNA Gene Duplication in Identical Twins Who Developed PSP

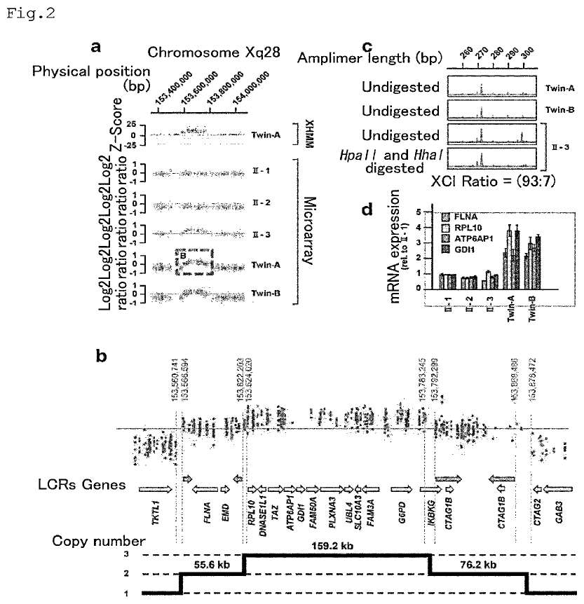

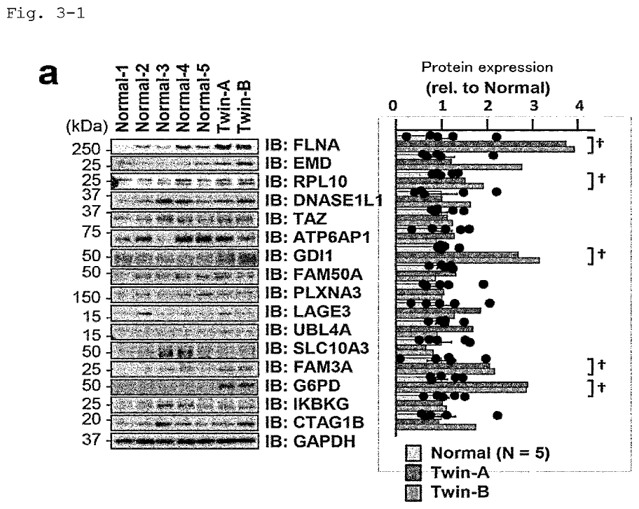

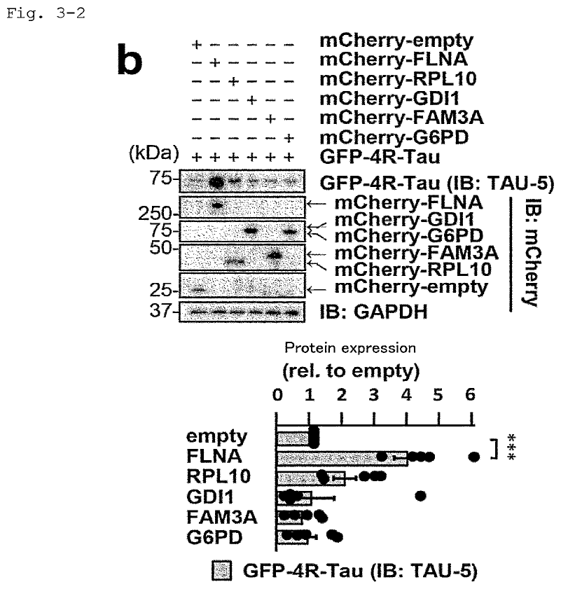

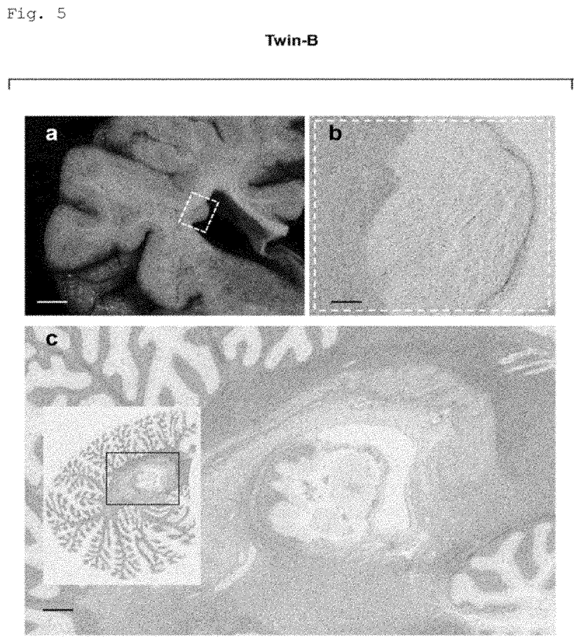

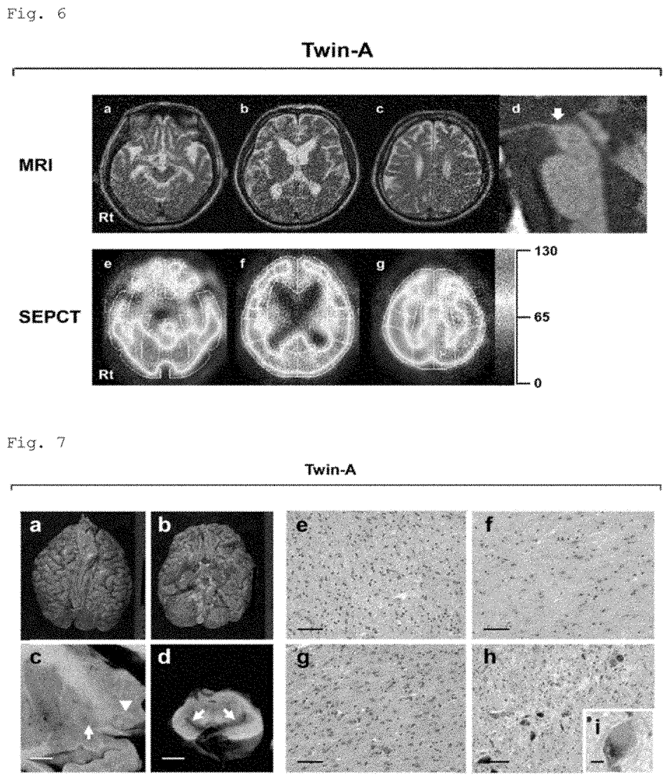

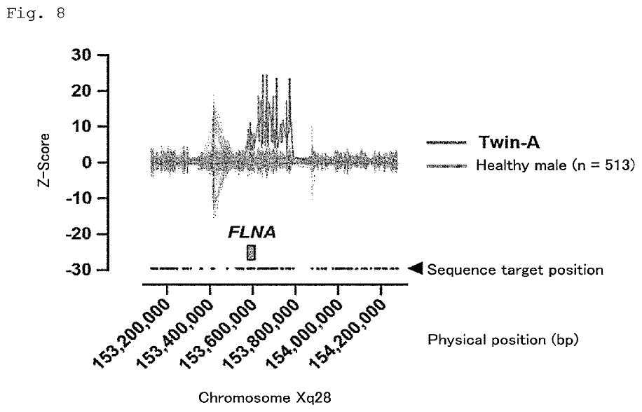

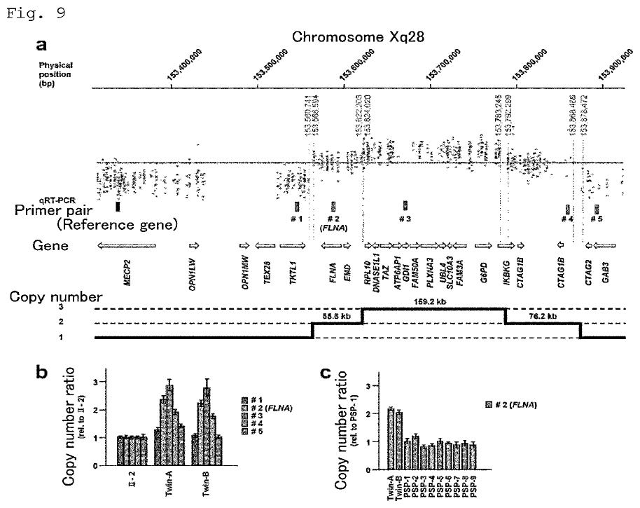

Attempts to implement the whole exome analysis first using XHMM, referred to as “exome-first” approach, have identified not only base sequence abnormalities but also copy number abnormalities in diseases of unknown cause (Reference Literature 28 to 31). Based on the hypothesis that the genomic abnormalities of the identical twins described above would be involved in PSP pathology, whole exome analysis of Twin-A was performed using XHMM. Although no abnormalities of the base sequence (including the MAPT gene) were identified, an abnormal copy number region (about 0.3 Mb in size) was detected in the Xq28 chromosomal region (FIG. 2a). No copy number abnormalities in this region were found in the 513 healthy Japanese males (FIG. 8). Next, chromosome microarrays were conducted on Twin-A, Twin-B, and the three siblings. As shown in FIG. 2b, a region with an increased number of copies was identified at 153.561 Mb to 153.878 Mb on chromosome Xq28 in Twin-A, Twin-B, and one non-affected female (II-3). The number of copies had a stepwise variation in the low-copy repeat (LCR) region, with 2 copies from 153.561 Mb to 153.878 Mb, 3 copies from 153.624 Mb to 153.783 Mb, and 2 copies from 153.792 Mb to 153.868 Mb. Changes in the number of copies were also confirmed by real-time quantitative PCR (FIG. 9). A study reports that there are families with a history of X-linked mental retardation with the same abnormal copy numbers at Xq28, and non-affected female carriers of such families show X chromosome inactivation (Reference Literature 19). In X chromosome inactivation analysis, non-affected female carrier II-3 showed a markedly skewed pattern, indicating that the abnormal X chromosome allele was inactivated by DNA methylation (FIG. 2c). Analysis of mRNA expression of immortalized lymphocytes was performed by real-time quantitative PCR. The mRNA expression levels of FLNA, RPL10, ATP6AP1, and GDI1 present in the abnormal copy number regions were more than twice as high in Twin-A and Twin-B compared with those of II-1 (control), while the mRNA expression levels in II-3 were comparable (FIG. 2d).

### Test Example 3: Filamin-A Promotes Phosphorylation, Protein Stabilization, and Sarkosyl Insolubility of 4R-Tau

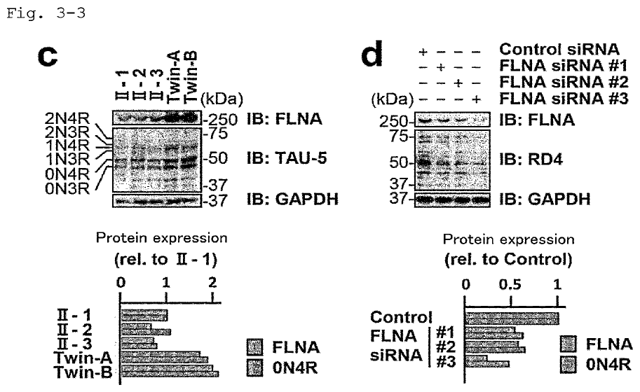

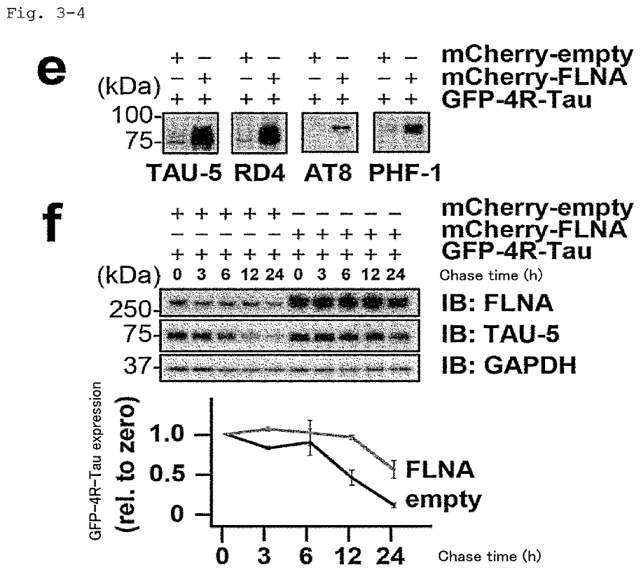

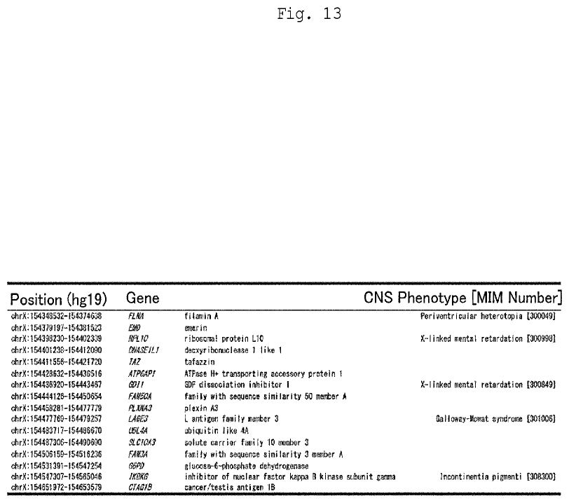

Sixteen different genes were encoded within the identified the abnormal copy number regions (FIG. 13). Western blotting of the frontal lobe revealed that the expression levels of filamin-A, RPL10, GDI1, FAM3A, and G6PD were increased in two cases (twins) compared with a healthy control group (Normal-1 to Normal-5) (FIG. 3a). To examine the effect of these five proteins on the pathology caused by 4R-tau, mCherry-tagged expression constructs of individual proteins were created and transferred together with a GFP-tagged wild-type 4R-tau construct (GFP-4R-tau) into HEK293 cells, followed by western blotting. The results reveled that, unlike the other four proteins, the protein expression level of GFP-4R-tau was statistically significantly increased when filamin-A was expressed (P<0.001, FIG. 3b). In the immortalized lymphocytes derived from the twins, not only the expression level of filamin-A but also the expression level of tau protein was increased (FIG. 3C). When three types of siRNAs targeting FLNA were introduced into immortalized lymphocytes derived from Twin-A, the expression level of not only filamin-A, but also tau protein, was decreased in all types of siRNAs (FIG. 3d). These results suggested that of the 16 genes in the abnormal copy number regions, FLNA would be a regulator for the expression of tau protein. Additionally, western blotting using AT8 and PHF-1, which are major phosphorylated tau antibodies, revealed that HEK293 cells expressing filamin-A and GFP-4R-tau showed increased phosphorylation of GFP-4R-tau (FIG. 3e). A cycloheximide chase assay was performed; the protein half-life of GFP-4R-tau introduced into HEK293 cells was prolonged when filamin-A was expressed (FIG. 3f). A soluble fraction (S1) and a sarkosyl-insoluble fraction (P3) were extracted from HEK293 cells expressing filamin-A and GFP-4R-tau. In both S1 and P3, the expression level of GFP-4R-tau was statistically significantly increased by the high expression of filamin-A (P<0.05, FIG. 3g). Immunoprecipitation was performed on HEK293 cells expressing filamin-A and GFP-4R-tau by using a tau antibody TAU-5, and the interaction of tau protein with filamin-A was identified (FIG. 3h). Tau protein also induced heat shock proteins HSP90, HSP70, HSP40, and ubiquitin when filamin-A was expressed, suggesting that the interaction between tau protein and filamin-A stresses the cells. In its protein structure, filamin-A has an actin-binding domain (ABD) at the N-terminus, and p.Ala39Gly mutation within the ABD is known to cause loss of its binding ability to filamentous actin (F-Actin) (Reference Literature 32). Unlike the wild type, the expression of p.Ala39Gly mutant filamin-A in HEK293 cells did not lead to an increased expression level of GFP-4R-tau protein, suggesting that filamin-A affects tau protein via F-Actin (FIG. 10).

### Test Example 4: A Compound that Inhibits the Expression of the Filamin-A Gene Decreases the Expression Level of Tau Protein

A test is performed in the same manner as in the test using siRNAs in Test Example 3, except that the siRNAs are replaced with antisense nucleic acids. The expression levels of tau protein as well as filamin-A can be decreased with the antisense nucleic acids.

### Test Example 5: Increased Expression Level of Filamin-A Protein and Colocalization of 4R-Tau in Autopsied PSP Brain

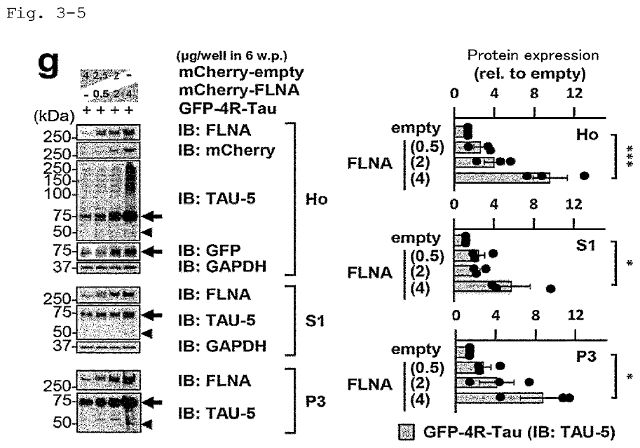

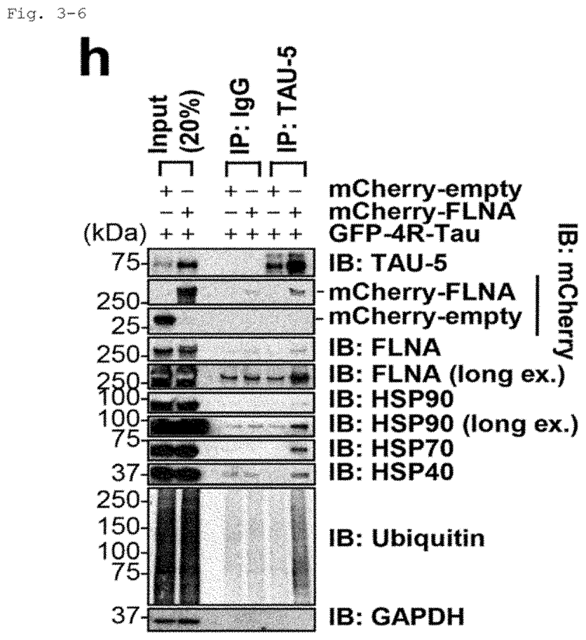

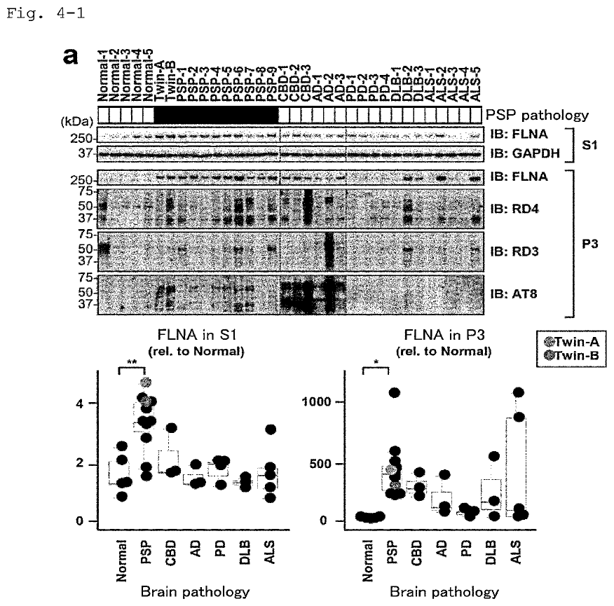

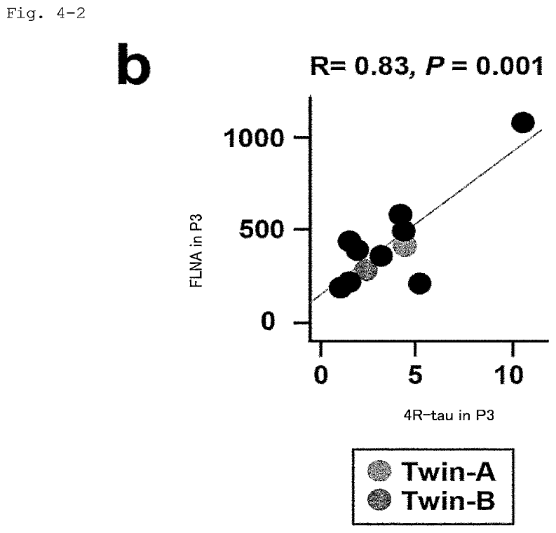

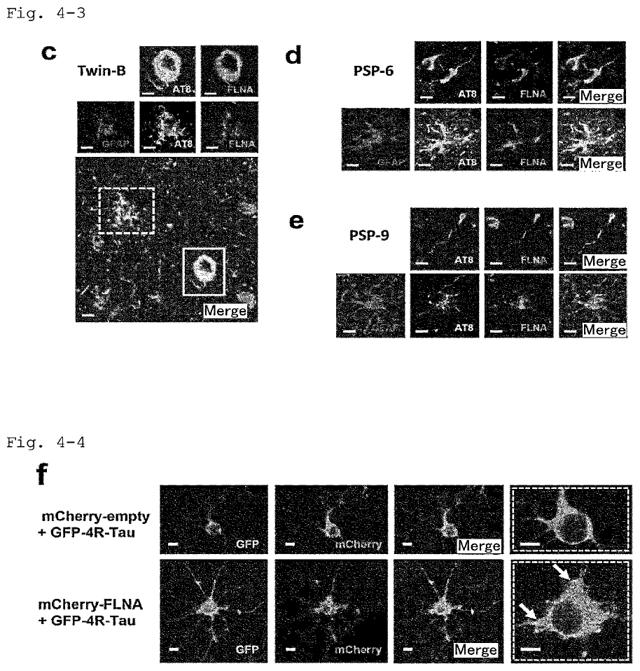

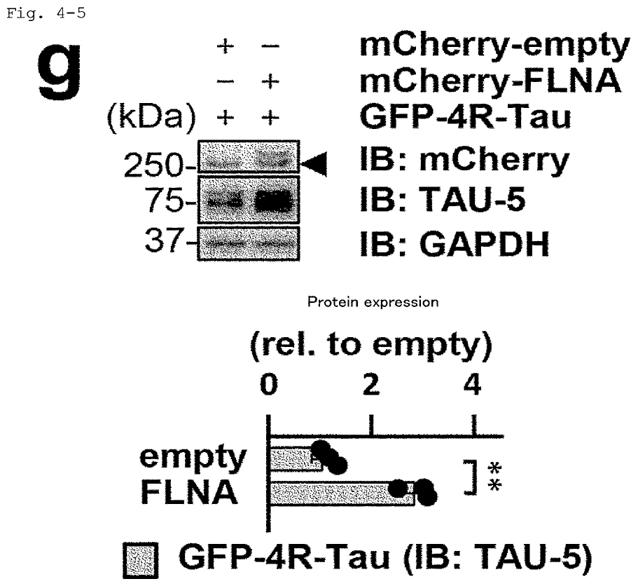

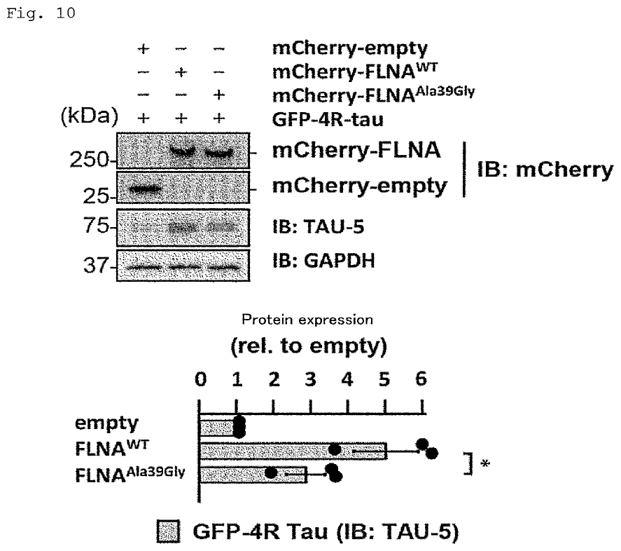

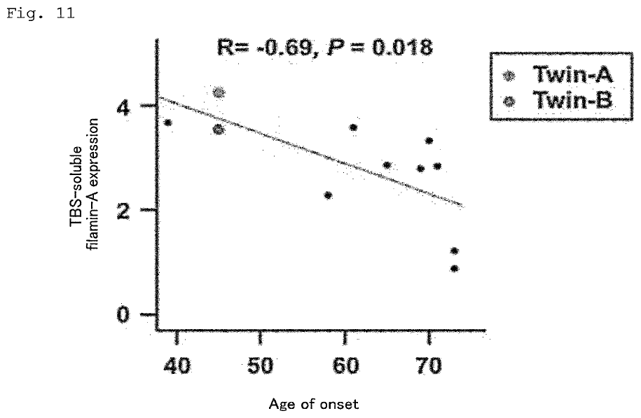

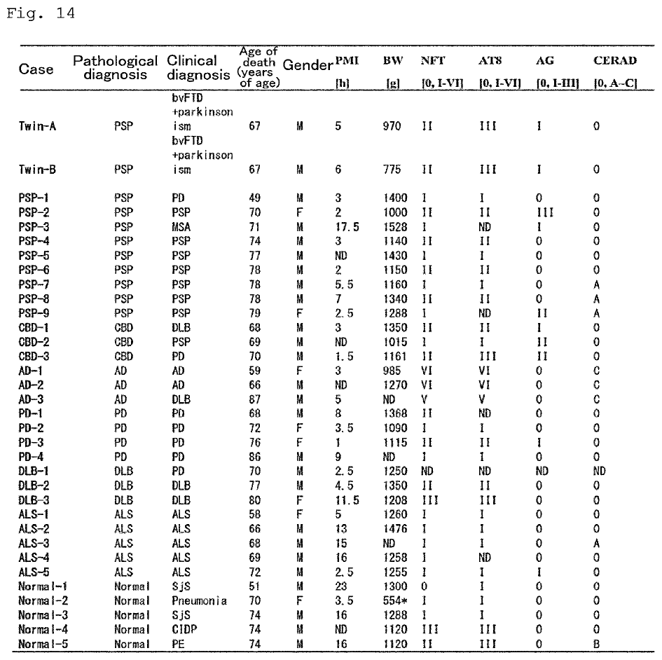

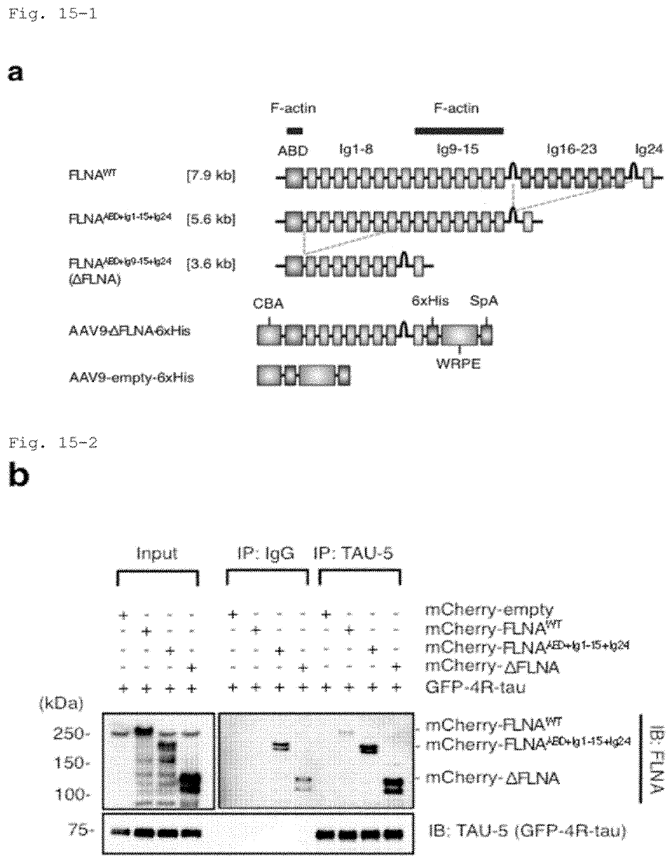

The expression level of filamin-A protein was analyzed by western blotting in autopsy brains of 32 cases in total: 11 cases of PSP (including Twin-A, Twin-B, and 9 sporadic cases), 3 cases of CBD, 3 cases of AD, 4 cases of PD, 3 cases of DLB, 5 cases of ALS, and 5 healthy controls. FIG. 14 shows details of the cases. The number of copies of the FLNA gene was analyzed by real-time quantitative PCR for the genomic DNA extracted from the autopsied PSP brains, and the analysis found no cases with an increased number of copies of the FLNA gene except for the twins (Twin-A and Twin-B) (FIG. 9). A study in the past reports that TA, which is a pathological characteristic of PSP, tends to appear in the frontal lobe (Reference Literature 33). Given this report, frontal lobes were sampled in this western blotting assay. The results revealed a statistically significant increase in the expression level of filamin-A protein in both the TBS-soluble fraction S1 and the sarkosyl-insoluble fraction P3 from the autopsied PSP brains (FIG. 4a). In S1, there was a statistically significant negative correlation between the filamin-A protein expression level and the age at onset of PSP (FIG. 11). In P3, there was a statistically significant positive correlation between the filamin-A protein expression level and the 4R-tau protein expression level (FIG. 4b). Fluorescent immunostaining of the frontal lobes of Twin-B (FIG. 4c) and sporadic PSP cases (FIGS. 4d and 4e) was performed; filamin-A colocalized with AT8-antibody-positive TA and globose-type NFT.

### Test Example 6: Filamin-A Expression Causes the Aggregation of 4R-Tau in Primary Astroglia

Filamin-A was expressed in the primary astroglia of a rat cerebral cortex, and the effect on aggregation of 4R-tau was examined. In primary astroglia co-expressing filamin-A and GFP-4R-tau, GFP-4R-tau was aggregated in the proximal area of the cell process and the cell body (FIG. 4f). The distribution of tau aggregation was similar to that of TA in PSP. Western blotting found that the expression of filamin-A statistically significantly increased the expression level of GFP-4R-tau in the primary astroglia (P<0.01, n=3).

### Test Example 7: Introduction of ΔFLNA Also Causes an Increase in the Expression Level and Phosphorylation of 4R-Tau

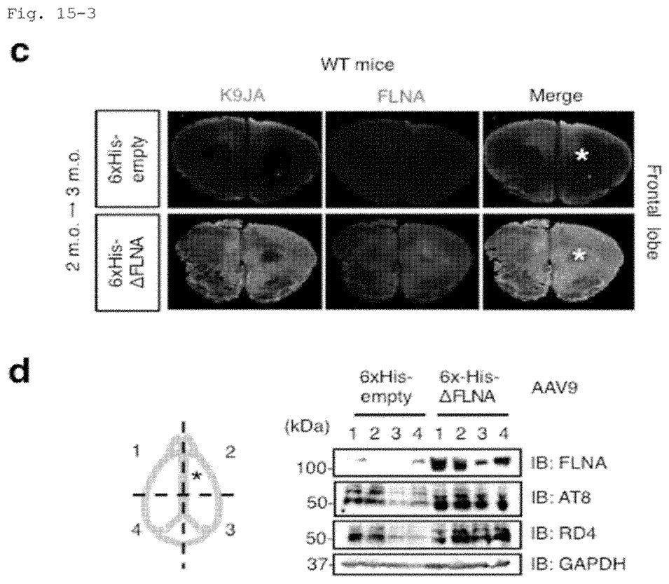

To examine the effect of ΔFLNA on the pathology caused by 4R-tau, an mCherry-tagged expression construct was prepared and transferred together with a GFP-tagged wild-type 4R-tau construct (GFP-4R-tau) into HEK293 cells, followed by western blotting. As a result, the expression level of GFP-4R-tau was increased when ΔFLNA was expressed (FIG. 15b). To examine the effect in vivo, AAV9-ΔFLNA-6×His was injected into the right frontal lobe of a two-month-old wild-type (WT) and into the right frontal lobe of a two-month-old transgenic mouse that expresses tau protein of humans (hT-PAC-N), and the mice were analyzed at 3 months of age. The results revealed an increased expression level of 4R-tau (FIGS. 15c to 15f).

### Test Example 8: The Expression Level of 4R-Tau is Increased in Transgenic Mice in which the Expression of Human FLNA is Induced (hFLNA-Tg)

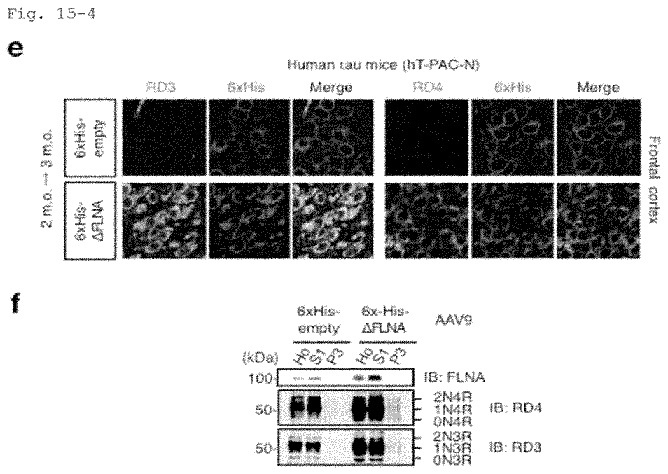

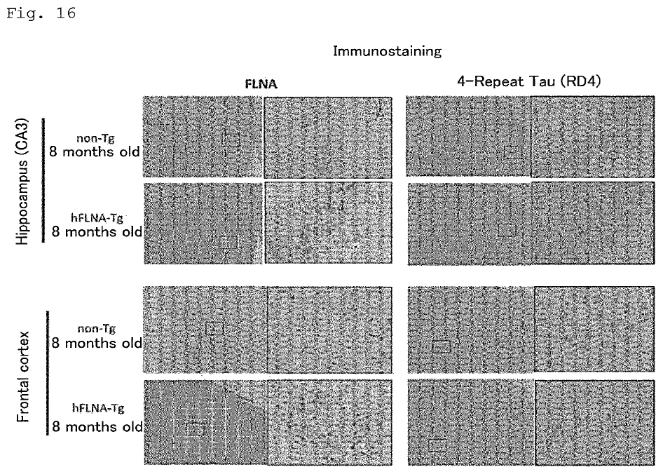

An 8-month-old transgenic mouse (hFLNA-Tg) was analyzed. In the hippocampus and frontal cortex, increased expression levels of FLNA and 4-repeat tau were observed (FIG. 16).

### Test Example 9: Mutant FLNA with Loss of Actin Binding does not Cause Gray Matter Heterotopia

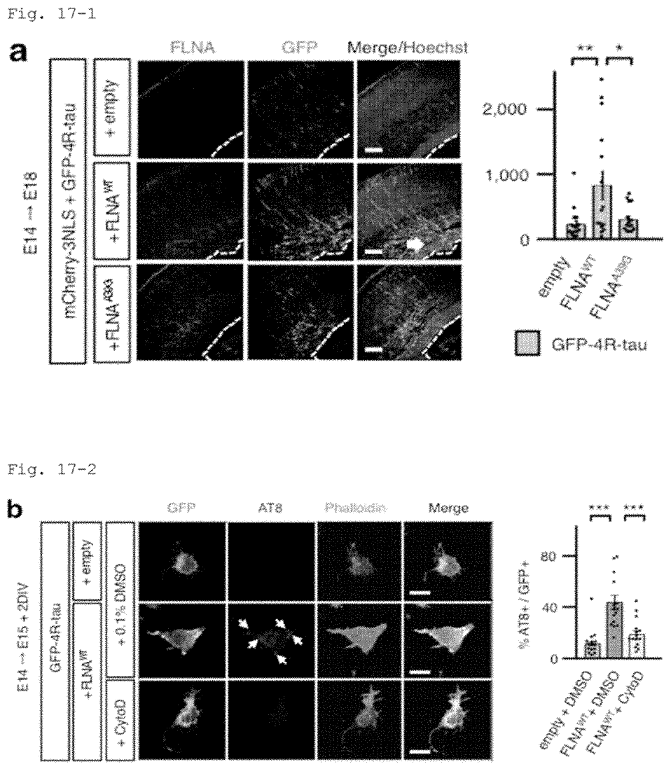

A mutant FLNA gene with loss of actin binding was introduced into the brain of a fetal mouse in a mouse at 14 days of gestation (E14). The mouse did not show gray matter heterotopia and an increased expression level of 4R-tau (FIG. 17a).

### Test Example 10: Effect of Actin Polymerization Inhibitor

The FLNA gene was induced into the brain of a fetal mouse in a mouse at 14 days of gestation (E14). Compared with 0.1% DMSO administration, the administration of an actin polymerization inhibitor, cytochalasin D (CytoD), did not cause an increase in the expression level of phosphorylated tau (FIG. 17b).

### 3. Discussion

The present study revealed that filamin-A promotes the aggregation of 4R-tau and is involved in the pathology of PSP. Identical twins without mutation in the MAPT gene developed PSP at the same time, and duplications of the FLNA gene encoding filamin-A were identified in their genomic DNA. Pathological analysis of brain bank samples revealed that the expression levels of filamin-A were increased in autopsied PSP brains, and that increased filamin-A colocalized with aggregated tau protein. Biochemical analysis further revealed that filamin-A promotes the phosphorylation, protein stabilization, and sarkosyl insolubility of 4R-tau, which are pre-stages of aggregation of 4R-tau, suggesting that filamin-A may be located upstream of 4R-tau in the pathology of PSP.

### REFERENCE LITERATURE

1. Boxer A L, Yu J T, Golbe L I, Litvan I, Lang A E, Hoglinger G U. Advances in progressive supranuclear palsy: new diagnostic criteria, biomarkers, and therapeutic approaches. Lancet Neurol 2017; 16: 552-63.
2. Glasmacher S A, Leigh P N, Saha R A. Predictors of survival in progressive supranuclear palsy and multiple system atrophy: a systematic review and meta-analysis. J Neurol Neurosurg Psychiatry 2017; 88: 402-11.
3. Uttl B, Santacruz P, Litvan I, Grafman J. Caregiving in progressive supranuclear palsy. Neurology 1998; 51: 1303-1309.
4. Yoshida M. Astrocytic inclusions in progressive supranuclear palsy and corticobasal degeneration. Neuropathology 2014; 34: 555-70.
5. Ishigaki S, Fujioka Y, Okada Y, Riku Y, Udagawa T, Honda D, et al. Altered Tau Isoform Ratio Caused by Loss of FUS and SFPQ Function Leads to FTLD-like Phenotypes. Cell Rep 2017; 18: 1118-31.
6. Fujioka S, Algom P A, Murray M E, Strongosky A, Soto-Ortolaza A I, Rademakers R, et al. Similarities between familial and sporadic autopsy-proven progressive supranuclear palsy. Neurology 2013; 80: 2076-8
7. Litvan I, Hauw J J, Bartko J J, Lantos P L, Daniel S E, Horoupian D S, et al. Validity and reliability of the preliminary NINDS neuropathologic criteria for progressive supranuclear palsy and related disorders. J Neuropathol Exp Neurol 1996; 55: 97-105.
8. Dickson D W, Bergeron C, Chin S S, Duyckaerts C, Horoupian D, Ikeda K, et al. Office of Rare Diseases neuropathologic criteria for corticobasal degeneration. J Neuropathol Exp Neurol 2002; 61: 935-46.
9. Montine T J, Phelps C H, Beach T G, Bigio E H, Cairns N J, Dickson D W, et al. National Institute on Aging-Alzheimer's Association guidelines for the neuropathologic assessment of Alzheimer's disease: a practical approach. Acta Neuropathol 2012; 123: 1-11.
10. Gelb D J, Oliver E, Gilman S. Diagnostic criteria for Parkinson disease. Arch Neural 1999; 56: 33-9.
11. McKeith I G, Dickson D W, Lowe J, Emre M, O'Brien J T, Feldman H, et al. Diagnosis and management of dementia with Lewy bodies: third report of the DLB Consortium. Neurology 2005; 65: 1863-72.
12. Cairns N J, Neumann M, Bigio E H, Holm I E, Troost D, Hatanpaa K J, et al. TDP-43 in familial and sporadic frontotemporal lobar degeneration with ubiquitin inclusions. Am J Pathol 2007; 171: 227-40.
13. Braak H, Braak E Neuropathological staging of Alzheimer-related changes. Acta Neuropathol 1991; 82: 239-59.
14. Braak H, Alafuzoff I, Arzberger T, Kretzschmar H, Del Tredici K. Staging of Alzheimer disease-associated neurofibrillary pathology using paraffin sections and immunocytochemistry. Acta Neuropathol 2006; 112: 389-404.
15. Saito Y, Ruberu N N, Sawabe M, Arai T, Tanaka N, Kakuta Y, et al. Staging of argyrophilic grains: an age-associated tauopathy. J Neuropathol Exp Neural 2004; 63: 911-18.
16. Mirra S S, Heyman A, McKeel D, Sumi S M, Crain B J, Brownlee L M, et al. The Consortium to Establish a Registry for Alzheimer's Disease (CERAD). Part I I. Standardization of the neuropathologic assessment of Alzheimer's disease. Neurology 1991; 41: 479-86.
17. Fromer M, Moran J L, Chambert K, Banks E, Bergen S E, Ruderfer D M, et al. Discovery and statistical genotyping of copy-number variation from whole-exome sequencing depth. Am J Hum Genet 2012; 91: 597-607.
18. Framer M, Purcell S M. Using XHMM Software to Detect Copy Number Variation in Whole-Exome Sequencing Data. Curr Protoc Hum Genet 2014; 81: 7.23.1-21.
19. Vandewalle J, Van Esch H, Govaerts K, Verbeeck J, Zweier C, Madrigal I, et al. Dosage-dependent severity of the phenotype in patients with mental retardation due to a recurrent copy-number gain at Xq28 mediated by an unusual recombination. Am J Hum Genet 2009; 85: 809-22.
20. Saitsu H, Nishimura T, Muramatsu K, Kodera H, Kumada S, Sugai K, et al. De novo mutations in the autophagy gene WDR45 cause static encephalopathy of childhood with neurodegeneration in adulthood. Nat Genet 2013; 45: 445-9.
21. Lim J, Balastik M, Lee T H, Nakamura K, Liou Y C, Sun A, et al. Pin1 has opposite effects on wild-type and P301L tau stability and tauopathy. J Clin Invest 2008; 118: 1877-89.
22. Sahara N, DeTure M, Ren Y, Ebrahim A S, Kang D, Knight J, et al. Characteristics of TBS-extractable hyperphosphorylated tau species: aggregation intermediates in rTg4510 mouse brain. J Alzheimers Dis 2013; 33: 249-63.
23. Chen Y, Balasubramaniyan V, Peng J, Hurlock E C, Tallquist M, Li J, et al. Isolation and culture of rat and mouse oligodendrocyte precursor cells. Nat Protoc 2007; 2: 1044-51.
24. Iida M, Katsuno M, Nakatsuji H, Adachi H, Kondo N, Miyazaki Y, et al. Pioglitazone suppresses neuronal and muscular degeneration caused by polyglutamine-expanded androgen receptors. Hum Mol Genet 2015; 24: 314-29.
25. Tsujikawa K, Hara K, Muro Y, Nakanishi H, Niwa Y, Koike M, et al. HMGCR antibody-associated myopathy as a paraneoplastic manifestation of esophageal carcinoma. Neurology 2016; 87: 841-3.
26. Piguet O, Hornberger M, Mioshi E, Hodges J R. Behavioural-variant frontotemporal dementia: diagnosis, clinical staging, and management. Lancet Neurol 2011; 10: 162-72.
27. Tsuboi Y, Slowinski J, Josephs K A, Honer W G, Wszolek Z K, Dickson D W. Atrophy of superior cerebellar peduncle in progressive supranuclear palsy. Neurology 2003; 60: 1766-9.
28. Miyatake S, Koshimizu E, Fujita A, Fukai R, Imagawa E, Ohba C, et al. Detecting copy-number variations in whole-exome sequencing data using the eXome Hidden Markov Model: an ‘exome-first’ approach. J Hum Genet 2015; 60: 175-82.
29. Nakashima M, Tohyama J, Nakagawa E, Watanabe Y, Siew C G, Kwong C S, et al. J Hum Genet 2019; 64: 313-22.
30. Tsuchida N, Nakashima M, Kato M, Heyman E, Inui T, Haginoya K, et al. Detection of copy number variations in epilepsy using exome data. Clin Genet 2018; 93: 577-87.
31. Chong P F, Saitsu H, Sakai Y, Imagi T, Nakamura R, Matsukura M, et al. Deletions of SCN2A and SCN3A genes in a patient with West syndrome and autistic spectrum disorder. Seizure 2018; 60: 91-3.
32. Iwamoto D V, Huehn A, Simon B, Huet-Calderwood C, Baldassarre M, Sindelar C V, et al. Structural basis of the filamin-A actin-binding domain interaction with F-actin. Nat Struct Mol Biol 2018; 25: 918-27.
33. Iwasaki Y, Yoshida M, Hattori M, Goto A, Aiba I, Hashizume Y, et al. Distribution of tuft-shaped astrocytes in the cerebral cortex in progressive supranuclear palsy. Acta Neuropathol 2004; 108: 399-405.
34. Aureliane Elie et al. Tau co-organizes dynamic microtubule and actin networks. Scientific Reports. 2015; 5:9964.
35. Yunior Cabrales Fontela et al. Multivalent crosslinking of actin filaments and microtubules through the microtubule-associated protein Tau. Nature Communications. 2017; 8:1981.

### INDUSTRIAL APPLICABILITY

The present invention provides a novel therapeutic strategy for PSP, for which no effective therapeutic methods and medicaments have been available. The medicament for PSP according to the present invention targets filamin-A and exerts its effect by a unique action mechanism. Additionally, cells expressing high levels of filamin-A (e.g., lymphocyte cell lines derived from a patient with PSP) or transgenic non-human mammals provided by the present invention are useful in research and development of medicaments and therapeutic methods for PSP. Specifically, the invention is further expected to lead to the creation of medicaments and therapeutic methods for PSP.

This invention is not limited in any way to the above description of the embodiments of the invention and the Examples. Various modified embodiments are also included in the invention to the extent that those skilled in the art would be able to readily conceive of without departing from the description of the claims. The content of the research papers, published patent applications, and published patents explicitly mentioned herein is incorporated by reference in their entirety.

1-2. (canceled)

3. A method for treating progressive supranuclear palsy in a subject, the method comprising:
administering a medicine for progressive supranuclear palsy comprising a compound for inhibiting expression of a filamin-A gene to a subject.

4. A method for assessing efficacy of a test substance on progressive supranuclear palsy, the method comprising:
(i) bringing a test substance into contact with a cell expressing filamin-A; and
(ii) detecting an expression of filamin-A, an amount of 4-repeat tau, and/or an amount of phosphorylated tau in the cell to determine efficacy of the test substance based on detection results,
wherein a decreased expression level of filamin-A, a decreased amount of 4-repeat tau, and/or a decreased amount of phosphorylated tau is an indicator of efficacy of the test substance.

5. The method according of claim 4, wherein the cell is a lymphocyte cell line derived from a patient with progressive supranuclear palsy.

6. A lymphocyte cell line, derived from a patient with progressive supranuclear palsy,
wherein the lymphocyte cell line has an increased expression level of filamin-A.

7. A non-human mammal having a high expression of filamin-A due to introduction of a filamin-A gene,
wherein the mammal presents a progressive supranuclear palsy-like pathology.

8. The mammal of claim 7, which is a transgenic animal.

9. The mammal of claim 7, wherein the progressive supranuclear palsy-like pathology is increased 4-repeat tau and/or increased phosphorylated tau in a neuron and/or a glial cell.

10. The mammal of claim 7, wherein the non-human mammal belongs to a species (genus) selected from the group consisting of mice, rats, guinea pigs, hamsters, rabbits, dogs, cats, and monkeys.

11. The mammal of claim 7, wherein to a species (genus) of mice.

12. The method of claim 3, wherein the compound is selected from the group consisting of:
(a) an siRNA targeting the filamin-A gene;
(b) a nucleic acid construct intracellularly forming an siRNA targeting the filamin-A gene;
(c) a single-stranded RNA containing an expression suppression sequence inhibiting expression of the filamin-A gene and a complementary sequence annealing to the expression suppression sequence;
(d) an antisense nucleic acid targeting a transcript of the filamin-A gene; and
(e) a ribozyme targeting a transcript of the filamin-A gene.

13. The method of claim 3, wherein the compound comprises an siRNA targeting the filamin-A gene.

14. The method of claim 3, wherein the compound comprises a nucleic acid construct intracellularly forming an siRNA targeting the filamin-A gene.

15. The method of claim 3, wherein the compound comprises a single-stranded RNA containing an expression suppression sequence inhibiting expression of the filamin-A gene and a complementary sequence annealing to the expression suppression sequence.

16. The method of claim 3, wherein the compound comprises an antisense nucleic acid targeting a transcript of the filamin-A gene.

17. The method of claim 3, wherein the compound comprises a ribozyme targeting a transcript of the filamin-A gene.


SEQUENCE LISTING (15 sequences)

SEQ ID NO: 1 - protein, 2639 aa, Homo sapiens
MSSSHSRAGQSAAGAAPGGGVDTRDAEMPATEKDLAEDAPWKKIQQNTFTRWCNEHLKCVSKRIANLQTDLSDGLRLIALLEVLSQKKMHRKHNQRPTFRQMQLENVSVALEFLDRESIKLVSIDSKAIVDGNLKLILGLIWTLILHYSISMPMWDEEEDEEAKKQTPKQRLLGWIQNKLPQLPITNFSRDWQSGRALGALVDSCAPGLCPDWDSWDASKPVTNAREAMQQADDWLGIPQVITPEEIVDPNVDEHSVMTYLSQFPKAKLKPGAPLRPKLNPKKARAYGPGIEPTGNMVKKRAEFTVETRSAGQGEVLVYVEDPAGHQEEAKVTANNDKNRTFSVWYVPEVTGTHKVTVLFAGQHIAKSPFEVYVDKSQGDASKVTAQGPGLEPSGNIANKTTYFEIFTAGAGTGEVEVVIQDPMGQKGTVEPQLEARGDSTYRCSYQPTMEGVHTVHVTFAGVPIPRSPYTVTVGQACNPSACRAVGRGLQPKGVRVKETADFKVYTKGAGSGELKVTVKGPKGEERVKQKDLGDGVYGFEYYPMVPGTYIVTITWGGQNIGRSPFEVKVGTECGNQKVRAWGPGLEGGVVGKSADFVVEAIGDDVGTLGFSVEGPSQAKIECDDKGDGSCDVRYWPQEAGEYAVHVLCNSEDIRLSPFMADIRDAPQDFHPDRVKARGPGLEKTGVAVNKPAEFTVDAKHGGKAPLRVQVQDNEGCPVEALVKDNGNGTYSCSYVPRKPVKHTAMVSWGGVSIPNSPFRVNVGAGSHPNKVKVYGPGVAKTGLKAHEPTYFTVDCAEAGQGDVSIGIKCAPGVVGPAEADIDFDIIRNDNDTFTVKYTPRGAGSYTIMVLFADQATPTSPIRVKVEPSHDASKVKAEGPGLSRTGVELGKPTHFTVNAKAAGKGKLDVQFSGLTKGDAVRDVDIIDHHDNTYTVKYTPVQQGPVGVNVTYGGDPIPKSPFSVAVSPSLDLSKIKVSGLGEKVDVGKDQEFTVKSKGAGGQGKVASKIVGPSGAAVPCKVEPGLGADNSVVRFLPREEGPYEVEVTYDGVPVPGSPFPLEAVAPTKPSKVKAFGPGLQGGSAGSPARFTIDTKGAGTGGLGLTVEGPCEAQLECLDNGDGTCSVSYVPTEPGDYNINILFADTHIPGSPFKAHVVPCFDASKVKCSGPGLERATAGEVGQFQVDCSSAGSAELTIEICSEAGLPAEVYIQDHGDGTHTITYIPLCPGAYTVTIKYGGQPVPNFPSKLQVEPAVDTSGVQCYGPGIEGQGVFREATTEFSVDARALTQTGGPHVKARVANPSGNLTETYVQDRGDGMYKVEYTPYEEGLHSVDVTYDGSPVPSSPFQVPVTEGCDPSRVRVHGPGIQSGTTNKPNKFTVETRGAGTGGLGLAVEGPSEAKMSCMDNKDGSCSVEYIPYEAGTYSLNVTYGGHQVPGSPFKVPVHDVTDASKVKCSGPGLSPGMVRANLPQSFQVDTSKAGVAPLQVKVQGPKGLVEPVDVVDNADGTQTVNYVPSREGPYSISVLYGDEEVPRSPFKVKVLPTHDASKVKASGPGLNTTGVPASLPVEFTIDAKDAGEGLLAVQITDPEGKPKKTHIQDNHDGTYTVAYVPDVTGRYTILIKYGGDEIPFSPYRVRAVPTGDASKCTVTGAGIGPTIQIGEETVITVDTKAAGKGKVTCTVCTPDGSEVDVDVVENEDGTFDIFYTAPQPGKYVICVRFGGEHVPNSPFQVTALAGDQPSVQPPLRSQQLAPQYTYAQGGQQTWAPERPLVGVNGLDVTSLRPFDLVIPFTIKKGEITGEVRMPSGKVAQPTITDNKDGTVTVRYAPSEAGLHEMDIRYDNMHIPGSPLQFYVDYVNCGHVTAYGPGLTHGVVNKPATFTVNTKDAGEGGLSLAIEGPSKAEISCTDNQDGTCSVSYLPVLPGDYSILVKYNEQHVPGSPFTARVTGDDSMRMSHLKVGSAADIPINISETDLSLLTATVVPPSGREEPCLLKRLRNGHVGISFVPKETGEHLVHVKKNGQHVASSPIPVVISQSEIGDASRVRVSGQGLHEGHTFEPAEFIIDTRDAGYGGLSLSIEGPSKVDINTEDLEDGTCRVTYCPTEPGNYIINIKFADQHVPGSPFSVKVTGEGRVKESITRRRRAPSVANVGSHCDLSLKIPEISIQDMTAQVTSPSGKTHEAEIVEGENHTYCIRFVPAEMGTHTVSVKYKGQHVPGSPFQFTVGPLGEGGAHKVRAGGPGLERAEAGVPAEFSIWTREAGAGGLAIAVEGPSKAEISFEDRKDGSCGVAYVVQEPGDYEVSVKFNEEHIPDSPFVVPVASPSGDARRLTVSSLQESGLKVNQPASFAVSLNGAKGAIDAKVHSPSGALEECYVTEIDQDKYAVRFIPRENGVYLIDVKFNGTHIPGSPFKIRVGEPGHGGDPGLVSAYGAGLEGGVTGNPAEFVVNTSNAGAGALSVTIDGPSKVKMDCQECPEGYRVTYTPMAPGSYLISIKYGGPYHIGGSPFKAKVTGPRLVSNHSLHETSSVFVDSLTKATCAPQHGAPGPGPADASKVVAKGLGLSKAYVGQKSSFTVDCSKAGNNMLLVGVHGPRTPCEEILVKHVGSRLYSVSYLLKDKGEYTLVVKWGDEHIPGSPYRVVVP

SEQ ID NO: 2 - DNA, 8533 bp, Homo sapiens
attcgcgtggaggcgcgtcgcgcgcagcggacgccgacagaatccccgaggcgcctggcgcgggcgcgggcgcgaaggcgatccgggcgccaccccgcggtcatcggtcaccggtcgctctcaggaacagcagcgcaacctctgctccctgcctcgcctcccgcgcgcctaggtgcctgcgactttaattaaagggccgtcccctcgccgaggctgcagcaccgcccccccggcttctcgcgcctcaaaatgagtagctcccactctcgggcgggccagagcgcagcaggcgcggctccgggcggcggcgtcgacacgcgggacgccgagatgccggccaccgagaaggacctggcggaggacgcgccgtggaagaagatccagcagaacactttcacgcgctggtgcaacgagcacctgaagtgcgtgagcaagcgcatcgccaacctgcagacggacctgagcgacgggctgcggcttatcgcgctgttggaggtgctcagccagaagaagatgcaccgcaagcacaaccagcggcccactttccgccaaatgcagcttgagaacgtgtcggtggcgctcgagttcctggaccgcgagagcatcaaactggtgtccatcgacagcaaggccatcgtggacgggaacctgaagctgatcctgggcctcatctggaccctgatcctgcactactccatctccatgcccatgtgggacgaggaggaggatgaggaggccaagaagcagacccccaagcagaggctcctgggctggatccagaacaagctgccgcagctgcccatcaccaacttcagccgggactggcagagcggccgggccctgggcgccctggtggacagctgtgccccgggcctgtgtcctgactgggactcttgggacgccagcaagcccgttaccaatgcgcgagaggccatgcagcaggcggatgactggctgggcatcccccaggtgatcacccccgaggagattgtggaccccaacgtggacgagcactctgtcatgacctacctgtcccagttccccaaggccaagctgaagccaggggctcccttgcggcccaaactgaacccgaagaaagcccgtgcctacgggccaggcatcgagcccacaggcaacatggtgaagaagcgggcagagttcactgtggagaccagaagtgctggccagggagaggtgctggtgtacgtggaggacccggccggacaccaggaggaggcaaaagtgaccgccaataacgacaagaaccgcaccttctccgtctggtacgtccccgaggtgacggggactcataaggttactgtgctctttgctggccagcacatcgccaagagccccttcgaggtgtacgtggataagtcacagggtgacgccagcaaagtgacagcccaaggtcccggcctggagcccagtggcaacatcgccaacaagaccacctactttgagatctttacggcaggagctggcacgggcgaggtcgaggttgtgatccaggaccccatgggacagaagggcacggtagagcctcagctggaggcccggggcgacagcacataccgctgcagctaccagcccaccatggagggcgtccacaccgtgcacgtcacgtttgccggcgtgcccatccctcgcagcccctacactgtcactgttggccaagcctgtaacccgagtgcctgccgggcggttggccggggcctccagcccaagggtgtgcgggtgaaggagacagctgacttcaaggtgtacacaaagggcgctggcagtggggagctgaaggtcaccgtgaagggccccaagggagaggagcgcgtgaagcagaaggacctgggggatggcgtgtatggcttcgagtattaccccatggtccctggaacctatatcgtcaccatcacgtggggtggtcagaacatcgggcgcagtcccttcgaagtgaaggtgggcaccgagtgtggcaatcagaaggtacgggcctggggccctgggctggagggcggcgtcgttggcaagtcagcagactttgtggtggaggctatcggggacgacgtgggcacgctgggcttctcggtggaagggccatcgcaggctaagatcgaatgtgacgacaagggcgacggctcctgtgatgtgcgctactggccgcaggaggctggcgagtatgccgttcacgtgctgtgcaacagcgaagacatccgcctcagccccttcatggctgacatccgtgacgcgccccaggacttccacccagacagggtgaaggcacgtgggcctggattggagaagacaggtgtggccgtcaacaagccagcagagttcacagtggatgccaagcacggtggcaaggccccacttcgggtccaagtccaggacaatgaaggctgccctgtggaggcgttggtcaaggacaacggcaatggcacttacagctgctcctacgtgcccaggaagccggtgaagcacacagccatggtgtcctggggaggcgtcagcatccccaacagccccttcagggtgaatgtgggagctggcagccaccccaacaaggtcaaagtatacggccccggagtagccaagacagggctcaaggcccacgagcccacctacttcactgtggactgcgccgaggctggccagggggacgtcagcatcggcatcaagtgtgcccctggagtggtaggccccgccgaagctgacatcgacttcgacatcatccgcaatgacaatgacaccttcacggtcaagtacacgccccggggggctggcagctacaccattatggtcctctttgctgaccaggccacgcccaccagccccatccgagtcaaggtggagccctctcatgacgccagtaaggtgaaggccgagggccctggcctcagtcgcactggtgtcgagcttggcaagcccacccacttcacagtaaatgccaaagctgctggcaaaggcaagctggacgtccagttctcaggactcaccaagggggatgcagtgcgagatgtggacatcatcgaccaccatgacaacacctacacagtcaagtacacgcctgtccagcagggtccagtaggcgtcaatgtcacttatggaggggatcccatccctaagagccctttctcagtggcagtatctccaagcctggacctcagcaagatcaaggtgtctggcctgggagagaaggtggacgttggcaaagaccaggagttcacagtcaaatcaaagggtgctggtggtcaaggcaaagtggcatccaagattgtgggcccctcgggtgcagcggtgccctgcaaggtggagccaggcctgggggctgacaacagtgtggtgcgcttcctgccccgtgaggaagggccctatgaggtggaggtgacctatgacggcgtgcccgtgcctggcagcccctttcctctggaagctgtggcccccaccaagcctagcaaggtgaaggcgtttgggccggggctgcagggaggcagtgcgggctcccccgcccgcttcaccatcgacaccaagggcgccggcacaggtggcctgggcctgacggtggagggcccctgtgaggcgcagctcgagtgcttggacaatggggatggcacatgttccgtgtcctacgtgcccaccgagcccggggactacaacatcaacatcctcttcgctgacacccacatccctggctccccattcaaggcccacgtggttccctgctttgacgcatccaaagtcaagtgctcaggccccgggctggagcgggccaccgctggggaggtgggccaattccaagtggactgctcgagcgcgggcagcgcggagctgaccattgagatctgctcggaggcggggcttccggccgaggtgtacatccaggaccacggtgatggcacgcacaccattacctacattcccctctgccccggggcctacaccgtcaccatcaagtacggcggccagcccgtgcccaacttccccagcaagctgcaggtggaacctgcggtggacacttccggtgtccagtgctatgggcctggtattgagggccagggtgtcttccgtgaggccaccactgagttcagtgtggacgcccgggctctgacacagaccggagggccgcacgtcaaggcccgtgtggccaacccctcaggcaacctgacggagacctacgttcaggaccgtggcgatggcatgtacaaagtggagtacacgccttacgaggagggactgcactccgtggacgtgacctatgacggcagtcccgtgcccagcagccccttccaggtgcccgtgaccgagggctgcgacccctcccgggtgcgtgtccacgggccaggcatccaaagtggcaccaccaacaagcccaacaagttcactgtggagaccaggggagctggcacgggcggcctgggcctggctgtagagggcccctccgaggccaagatgtcctgcatggataacaaggacggcagctgctcggtcgagtacatcccttatgaggctggcacctacagcctcaacgtcacctatggtggccatcaagtgccaggcagtcctttcaaggtccctgtgcatgatgtgacagatgcgtccaaggtcaagtgctctgggcccggcctgagcccaggcatggttcgtgccaacctccctcagtccttccaggtggacacaagcaaggctggtgtggccccattgcaggtcaaagtgcaagggcccaaaggcctggtggagccagtggacgtggtagacaacgctgatggcacccagaccgtcaattatgtgcccagccgagaagggccctacagcatctcagtactgtatggagatgaagaggtaccccggagccccttcaaggtcaaggtgctgcctactcatgatgccagcaaggtgaaggccagtggccccgggctcaacaccactggcgtgcctgccagcctgcccgtggagttcaccatcgatgcaaaggacgccggggagggcctgctggctgtccagatcacggatcccgaaggcaagccgaagaagacacacatccaagacaaccatgacggcacgtatacagtggcctacgtgccagacgtgacaggtcgctacaccatcctcatcaagtacggtggtgacgagatccccttctccccgtaccgcgtgcgtgccgtgcccaccggggacgccagcaagtgcactgtcacaggtgctggcatcggccccaccattcagattggggaggagacggtgatcactgtggacactaaggcggcaggcaaaggcaaagtgacgtgcaccgtgtgcacgcctgatggctcagaggtggatgtggacgtggtggagaatgaggacggcactttcgacatcttctacacggccccccagccgggcaaatacgtcatctgtgtgcgctttggtggcgagcacgtgcccaacagccccttccaagtgacggctctggctggggaccagccctcggtgcagccccctctacggtctcagcagctggccccacagtacacctacgcccagggcggccagcagacttgggccccggagaggcccctggtgggtgtcaatgggctggatgtgaccagcctgaggccctttgaccttgtcatccccttcaccatcaagaagggcgagatcacaggggaggttcggatgccctcaggcaaggtggcgcagcccaccatcactgacaacaaagacggcaccgtgaccgtgcggtatgcacccagcgaggctggcctgcacgagatggacatccgctatgacaacatgcacatcccaggaagccccttgcagttctatgtggattacgtcaactgtggccatgtcactgcctatgggcctggcctcacccatggagtagtgaacaagcctgccaccttcaccgtcaacaccaaggatgcaggagaggggggcctgtctctggccattgagggcccgtccaaagcagaaatcagctgcactgacaaccaggatgggacatgcagcgtgtcctacctgcctgtgctgccgggggactacagcattctagtcaagtacaatgaacagcacgtcccaggcagccccttcactgctcgggtcacaggtgacgactccatgcgtatgtcccacctaaaggtcggctctgctgccgacatccccatcaacatctcagagacggatctcagcctgctgacggccactgtggtcccgccctcgggccgggaggagccctgtttgctgaagcggctgcgtaatggccacgtggggatttcattcgtgcccaaggagacgggggagcacctggtgcatgtgaagaaaaatggccagcacgtggccagcagccccatcccggtggtgatcagccagtcggaaattggggatgccagtcgtgttcgggtctctggtcagggccttcacgaaggccacacctttgagcctgcagagtttatcattgatacccgcgatgcaggctatggtgggctcagcctgtccattgagggccccagcaaggtggacatcaacacagaggacctggaggacgggacgtgcagggtcacctactgccccacagagccaggcaactacatcatcaacatcaagtttgccgaccagcacgtgcctggcagccccttctctgtgaaggtgacaggcgagggccgggtgaaagagagcatcacccgcaggcgtcgggctccttcagtggccaacgttggtagtcattgtgacctcagcctgaaaatccctgaaattagcatccaggatatgacagcccaggtgaccagcccatcgggcaagacccatgaggccgagatcgtggaaggggagaaccacacctactgcatccgctttgttcccgctgagatgggcacacacacagtcagcgtgaagtacaagggccagcacgtgcctgggagccccttccagttcaccgtggggcccctaggggaagggggagcccacaaggtccgagctgggggccctggcctggagagagctgaagctggagtgccagccgaattcagtatctggacccgggaagctggtgctggaggcctggccattgctgtcgagggccccagcaaggctgagatctcttttgaggaccgcaaggacggctcctgtggtgtggcttatgtggtccaggagccaggtgactacgaagtctcagtcaagttcaacgaggaacacattcccgacagccccttcgtggtgcctgtggcttctccgtctggcgacgcccgccgcctcactgtttctagccttcaggagtcagggctaaaggtcaaccagccagcctcttttgcagtcagcctgaacggggccaagggggcgatcgatgccaaggtgcacagcccctcaggagccctggaggagtgctatgtcacagaaattgaccaagataagtatgctgtgcgcttcatccctcgggagaatggcgtttacctgattgacgtcaagttcaacggcacccacatccctggaagccccttcaagatccgagttggggagcctgggcatggaggggacccaggcttggtgtctgcttacggagcaggtctggaaggcggtgtcacagggaacccagctgagttcgtcgtgaacacgagcaatgcgggagctggtgccctgtcggtgaccattgacggcccctccaaggtgaagatggattgccaggagtgccctgagggctaccgcgtcacctatacccccatggcacctggcagctacctcatctccatcaagtacggcggcccctaccacattgggggcagccccttcaaggccaaagtcacaggcccccgtctcgtcagcaaccacagcctccacgagacatcatcagtgtttgtagactctctgaccaaggccacctgtgccccccagcatggggccccgggtcctgggcctgctgacgccagcaaggtggtggccaagggcctggggctgagcaaggcctacgtaggccagaagagcagcttcacagtagactgcagcaaagcaggcaacaacatgctgctggtgggggttcatggcccaaggaccccctgcgaggagatcctggtgaagcacgtgggcagccggctctacagcgtgtcctacctgctcaaggacaagggggagtacacactggtggtcaaatggggggacgagcacatcccaggcagcccctaccgcgttgtggtgccctgagtctggggcccgtgccagccggcagcccccaagcctgccccgctacccaagcagccccgccctcttcccctcaaccccggcccaggccgccctggccgcccgcctgtcactgcagccgcccctgccctgtgccgtgctgcgctcacctgcctccccagccagccgctgacctctcggctttcacttgggcagagggagccatttggtggcgctgcttgtcttctttggttctgggaggggtgagggatgggggtcctgtacacaaccacccactagttctcttctccagccaagaggaataaagttttgcttccattaaaaaaaaaaaaaaaaaaaaaaaaaaaaaaaaaaaaaaaaaaaaaaa

SEQ ID NO: 3 - protein, 2647 aa, Homo sapiens
MSSSHSRAGQSAAGAAPGGGVDTRDAEMPATEKDLAEDAPWKKIQQNTFTRWCNEHLKCVSKRIANLQTDLSDGLRLIALLEVLSQKKMHRKHNQRPTFRQMQLENVSVALEFLDRESIKLVSIDSKAIVDGNLKLILGLIWTLILHYSISMPMWDEEEDEEAKKQTPKQRLLGWIQNKLPQLPITNFSRDWQSGRALGALVDSCAPGLCPDWDSWDASKPVTNAREAMQQADDWLGIPQVITPEEIVDPNVDEHSVMTYLSQFPKAKLKPGAPLRPKLNPKKARAYGPGIEPTGNMVKKRAEFTVETRSAGQGEVLVYVEDPAGHQEEAKVTANNDKNRTFSVWYVPEVTGTHKVTVLFAGQHIAKSPFEVYVDKSQGDASKVTAQGPGLEPSGNIANKTTYFEIFTAGAGTGEVEVVIQDPMGQKGTVEPQLEARGDSTYRCSYQPTMEGVHTVHVTFAGVPIPRSPYTVTVGQACNPSACRAVGRGLQPKGVRVKETADFKVYTKGAGSGELKVTVKGPKGEERVKQKDLGDGVYGFEYYPMVPGTYIVTITWGGQNIGRSPFEVKVGTECGNQKVRAWGPGLEGGVVGKSADFVVEAIGDDVGTLGFSVEGPSQAKIECDDKGDGSCDVRYWPQEAGEYAVHVLCNSEDIRLSPFMADIRDAPQDFHPDRVKARGPGLEKTGVAVNKPAEFTVDAKHGGKAPLRVQVQDNEGCPVEALVKDNGNGTYSCSYVPRKPVKHTAMVSWGGVSIPNSPFRVNVGAGSHPNKVKVYGPGVAKTGLKAHEPTYFTVDCAEAGQGDVSIGIKCAPGVVGPAEADIDFDIIRNDNDTFTVKYTPRGAGSYTIMVLFADQATPTSPIRVKVEPSHDASKVKAEGPGLSRTGVELGKPTHFTVNAKAAGKGKLDVQFSGLTKGDAVRDVDIIDHHDNTYTVKYTPVQQGPVGVNVTYGGDPIPKSPFSVAVSPSLDLSKIKVSGLGEKVDVGKDQEFTVKSKGAGGQGKVASKIVGPSGAAVPCKVEPGLGADNSVVRFLPREEGPYEVEVTYDGVPVPGSPFPLEAVAPTKPSKVKAFGPGLQGGSAGSPARFTIDTKGAGTGGLGLTVEGPCEAQLECLDNGDGTCSVSYVPTEPGDYNINILFADTHIPGSPFKAHVVPCFDASKVKCSGPGLERATAGEVGQFQVDCSSAGSAELTIEICSEAGLPAEVYIQDHGDGTHTITYIPLCPGAYTVTIKYGGQPVPNFPSKLQVEPAVDTSGVQCYGPGIEGQGVFREATTEFSVDARALTQTGGPHVKARVANPSGNLTETYVQDRGDGMYKVEYTPYEEGLHSVDVTYDGSPVPSSPFQVPVTEGCDPSRVRVHGPGIQSGTTNKPNKFTVETRGAGTGGLGLAVEGPSEAKMSCMDNKDGSCSVEYIPYEAGTYSLNVTYGGHQVPGSPFKVPVHDVTDASKVKCSGPGLSPGMVRANLPQSFQVDTSKAGVAPLQVKVQGPKGLVEPVDVVDNADGTQTVNYVPSREGPYSISVLYGDEEVPRSPFKVKVLPTHDASKVKASGPGLNTTGVPASLPVEFTIDAKDAGEGLLAVQITDPEGKPKKTHIQDNHDGTYTVAYVPDVTGRYTILIKYGGDEIPFSPYRVRAVPTGDASKCTVTVSIGGHGLGAGIGPTIQIGEETVITVDTKAAGKGKVTCTVCTPDGSEVDVDVVENEDGTFDIFYTAPQPGKYVICVRFGGEHVPNSPFQVTALAGDQPSVQPPLRSQQLAPQYTYAQGGQQTWAPERPLVGVNGLDVTSLRPFDLVIPFTIKKGEITGEVRMPSGKVAQPTITDNKDGTVTVRYAPSEAGLHEMDIRYDNMHIPGSPLQFYVDYVNCGHVTAYGPGLTHGVVNKPATFTVNTKDAGEGGLSLAIEGPSKAEISCTDNQDGTCSVSYLPVLPGDYSILVKYNEQHVPGSPFTARVTGDDSMRMSHLKVGSAADIPINISETDLSLLTATVVPPSGREEPCLLKRLRNGHVGISFVPKETGEHLVHVKKNGQHVASSPIPVVISQSEIGDASRVRVSGQGLHEGHTFEPAEFIIDTRDAGYGGLSLSIEGPSKVDINTEDLEDGTCRVTYCPTEPGNYIINIKFADQHVPGSPFSVKVTGEGRVKESITRRRRAPSVANVGSHCDLSLKIPEISIQDMTAQVTSPSGKTHEAEIVEGENHTYCIRFVPAEMGTHTVSVKYKGQHVPGSPFQFTVGPLGEGGAHKVRAGGPGLERAEAGVPAEFSIWTREAGAGGLAIAVEGPSKAEISFEDRKDGSCGVAYVVQEPGDYEVSVKFNEEHIPDSPFVVPVASPSGDARRLTVSSLQESGLKVNQPASFAVSLNGAKGAIDAKVHSPSGALEECYVTEIDQDKYAVRFIPRENGVYLIDVKFNGTHIPGSPFKIRVGEPGHGGDPGLVSAYGAGLEGGVTGNPAEFVVNTSNAGAGALSVTIDGPSKVKMDCQECPEGYRVTYTPMAPGSYLISIKYGGPYHIGGSPFKAKVTGPRLVSNHSLHETSSVFVDSLTKATCAPQHGAPGPGPADASKVVAKGLGLSKAYVGQKSSFTVDCSKAGNNMLLVGVHGPRTPCEEILVKHVGSRLYSVSYLLKDKGEYTLVVKWGDEHIPGSPYRVVVP

SEQ ID NO: 4 - DNA, 8507 bp, Homo sapiens
gcgtggaggcgcgtcgcgcgcagcggacgccgacagaatccccgaggcgcctggcgcgggcgcgggcgcgaaggcgatccgggcgccaccccgcggtcatcggtcaccggtcgctctcaggaacagcagcgcaacctctgctccctgcctcgcctcccgcgcgcctaggtgcctgcgactttaattaaagggccgtcccctcgccgaggctgcagcaccgcccccccggcttctcgcgcctcaaaatgagtagctcccactctcgggcgggccagagcgcagcaggcgcggctccgggcggcggcgtcgacacgcgggacgccgagatgccggccaccgagaaggacctggcggaggacgcgccgtggaagaagatccagcagaacactttcacgcgctggtgcaacgagcacctgaagtgcgtgagcaagcgcatcgccaacctgcagacggacctgagcgacgggctgcggcttatcgcgctgttggaggtgctcagccagaagaagatgcaccgcaagcacaaccagcggcccactttccgccaaatgcagcttgagaacgtgtcggtggcgctcgagttcctggaccgcgagagcatcaaactggtgtccatcgacagcaaggccatcgtggacgggaacctgaagctgatcctgggcctcatctggaccctgatcctgcactactccatctccatgcccatgtgggacgaggaggaggatgaggaggccaagaagcagacccccaagcagaggctcctgggctggatccagaacaagctgccgcagctgcccatcaccaacttcagccgggactggcagagcggccgggccctgggcgccctggtggacagctgtgccccgggcctgtgtcctgactgggactcttgggacgccagcaagcccgttaccaatgcgcgagaggccatgcagcaggcggatgactggctgggcatcccccaggtgatcacccccgaggagattgtggaccccaacgtggacgagcactctgtcatgacctacctgtcccagttccccaaggccaagctgaagccaggggctcccttgcggcccaaactgaacccgaagaaagcccgtgcctacgggccaggcatcgagcccacaggcaacatggtgaagaagcgggcagagttcactgtggagaccagaagtgctggccagggagaggtgctggtgtacgtggaggacccggccggacaccaggaggaggcaaaagtgaccgccaataacgacaagaaccgcaccttctccgtctggtacgtccccgaggtgacggggactcataaggttactgtgctctttgctggccagcacatcgccaagagccccttcgaggtgtacgtggataagtcacagggtgacgccagcaaagtgacagcccaaggtcccggcctggagcccagtggcaacatcgccaacaagaccacctactttgagatctttacggcaggagctggcacgggcgaggtcgaggttgtgatccaggaccccatgggacagaagggcacggtagagcctcagctggaggcccggggcgacagcacataccgctgcagctaccagcccaccatggagggcgtccacaccgtgcacgtcacgtttgccggcgtgcccatccctcgcagcccctacactgtcactgttggccaagcctgtaacccgagtgcctgccgggcggttggccggggcctccagcccaagggtgtgcgggtgaaggagacagctgacttcaaggtgtacacaaagggcgctggcagtggggagctgaaggtcaccgtgaagggccccaagggagaggagcgcgtgaagcagaaggacctgggggatggcgtgtatggcttcgagtattaccccatggtccctggaacctatatcgtcaccatcacgtggggtggtcagaacatcgggcgcagtcccttcgaagtgaaggtgggcaccgagtgtggcaatcagaaggtacgggcctggggccctgggctggagggcggcgtcgttggcaagtcagcagactttgtggtggaggctatcggggacgacgtgggcacgctgggcttctcggtggaagggccatcgcaggctaagatcgaatgtgacgacaagggcgacggctcctgtgatgtgcgctactggccgcaggaggctggcgagtatgccgttcacgtgctgtgcaacagcgaagacatccgcctcagccccttcatggctgacatccgtgacgcgccccaggacttccacccagacagggtgaaggcacgtgggcctggattggagaagacaggtgtggccgtcaacaagccagcagagttcacagtggatgccaagcacggtggcaaggccccacttcgggtccaagtccaggacaatgaaggctgccctgtggaggcgttggtcaaggacaacggcaatggcacttacagctgctcctacgtgcccaggaagccggtgaagcacacagccatggtgtcctggggaggcgtcagcatccccaacagccccttcagggtgaatgtgggagctggcagccaccccaacaaggtcaaagtatacggccccggagtagccaagacagggctcaaggcccacgagcccacctacttcactgtggactgcgccgaggctggccagggggacgtcagcatcggcatcaagtgtgcccctggagtggtaggccccgccgaagctgacatcgacttcgacatcatccgcaatgacaatgacaccttcacggtcaagtacacgccccggggggctggcagctacaccattatggtcctctttgctgaccaggccacgcccaccagccccatccgagtcaaggtggagccctctcatgacgccagtaaggtgaaggccgagggccctggcctcagtcgcactggtgtcgagcttggcaagcccacccacttcacagtaaatgccaaagctgctggcaaaggcaagctggacgtccagttctcaggactcaccaagggggatgcagtgcgagatgtggacatcatcgaccaccatgacaacacctacacagtcaagtacacgcctgtccagcagggtccagtaggcgtcaatgtcacttatggaggggatcccatccctaagagccctttctcagtggcagtatctccaagcctggacctcagcaagatcaaggtgtctggcctgggagagaaggtggacgttggcaaagaccaggagttcacagtcaaatcaaagggtgctggtggtcaaggcaaagtggcatccaagattgtgggcccctcgggtgcagcggtgccctgcaaggtggagccaggcctgggggctgacaacagtgtggtgcgcttcctgccccgtgaggaagggccctatgaggtggaggtgacctatgacggcgtgcccgtgcctggcagcccctttcctctggaagctgtggcccccaccaagcctagcaaggtgaaggcgtttgggccggggctgcagggaggcagtgcgggctcccccgcccgcttcaccatcgacaccaagggcgccggcacaggtggcctgggcctgacggtggagggcccctgtgaggcgcagctcgagtgcttggacaatggggatggcacatgttccgtgtcctacgtgcccaccgagcccggggactacaacatcaacatcctcttcgctgacacccacatccctggctccccattcaaggcccacgtggttccctgctttgacgcatccaaagtcaagtgctcaggccccgggctggagcgggccaccgctggggaggtgggccaattccaagtggactgctcgagcgcgggcagcgcggagctgaccattgagatctgctcggaggcggggcttccggccgaggtgtacatccaggaccacggtgatggcacgcacaccattacctacattcccctctgccccggggcctacaccgtcaccatcaagtacggcggccagcccgtgcccaacttccccagcaagctgcaggtggaacctgcggtggacacttccggtgtccagtgctatgggcctggtattgagggccagggtgtcttccgtgaggccaccactgagttcagtgtggacgcccgggctctgacacagaccggagggccgcacgtcaaggcccgtgtggccaacccctcaggcaacctgacggagacctacgttcaggaccgtggcgatggcatgtacaaagtggagtacacgccttacgaggagggactgcactccgtggacgtgacctatgacggcagtcccgtgcccagcagccccttccaggtgcccgtgaccgagggctgcgacccctcccgggtgcgtgtccacgggccaggcatccaaagtggcaccaccaacaagcccaacaagttcactgtggagaccaggggagctggcacgggcggcctgggcctggctgtagagggcccctccgaggccaagatgtcctgcatggataacaaggacggcagctgctcggtcgagtacatcccttatgaggctggcacctacagcctcaacgtcacctatggtggccatcaagtgccaggcagtcctttcaaggtccctgtgcatgatgtgacagatgcgtccaaggtcaagtgctctgggcccggcctgagcccaggcatggttcgtgccaacctccctcagtccttccaggtggacacaagcaaggctggtgtggccccattgcaggtcaaagtgcaagggcccaaaggcctggtggagccagtggacgtggtagacaacgctgatggcacccagaccgtcaattatgtgcccagccgagaagggccctacagcatctcagtactgtatggagatgaagaggtaccccggagccccttcaaggtcaaggtgctgcctactcatgatgccagcaaggtgaaggccagtggccccgggctcaacaccactggcgtgcctgccagcctgcccgtggagttcaccatcgatgcaaaggacgccggggagggcctgctggctgtccagatcacggatcccgaaggcaagccgaagaagacacacatccaagacaaccatgacggcacgtatacagtggcctacgtgccagacgtgacaggtcgctacaccatcctcatcaagtacggtggtgacgagatccccttctccccgtaccgcgtgcgtgccgtgcccaccggggacgccagcaagtgcactgtcacagtgtcaatcggaggtcacgggctaggtgctggcatcggccccaccattcagattggggaggagacggtgatcactgtggacactaaggcggcaggcaaaggcaaagtgacgtgcaccgtgtgcacgcctgatggctcagaggtggatgtggacgtggtggagaatgaggacggcactttcgacatcttctacacggccccccagccgggcaaatacgtcatctgtgtgcgctttggtggcgagcacgtgcccaacagccccttccaagtgacggctctggctggggaccagccctcggtgcagccccctctacggtctcagcagctggccccacagtacacctacgcccagggcggccagcagacttgggccccggagaggcccctggtgggtgtcaatgggctggatgtgaccagcctgaggccctttgaccttgtcatccccttcaccatcaagaagggcgagatcacaggggaggttcggatgccctcaggcaaggtggcgcagcccaccatcactgacaacaaagacggcaccgtgaccgtgcggtatgcacccagcgaggctggcctgcacgagatggacatccgctatgacaacatgcacatcccaggaagccccttgcagttctatgtggattacgtcaactgtggccatgtcactgcctatgggcctggcctcacccatggagtagtgaacaagcctgccaccttcaccgtcaacaccaaggatgcaggagaggggggcctgtctctggccattgagggcccgtccaaagcagaaatcagctgcactgacaaccaggatgggacatgcagcgtgtcctacctgcctgtgctgccgggggactacagcattctagtcaagtacaatgaacagcacgtcccaggcagccccttcactgctcgggtcacaggtgacgactccatgcgtatgtcccacctaaaggtcggctctgctgccgacatccccatcaacatctcagagacggatctcagcctgctgacggccactgtggtcccgccctcgggccgggaggagccctgtttgctgaagcggctgcgtaatggccacgtggggatttcattcgtgcccaaggagacgggggagcacctggtgcatgtgaagaaaaatggccagcacgtggccagcagccccatcccggtggtgatcagccagtcggaaattggggatgccagtcgtgttcgggtctctggtcagggccttcacgaaggccacacctttgagcctgcagagtttatcattgatacccgcgatgcaggctatggtgggctcagcctgtccattgagggccccagcaaggtggacatcaacacagaggacctggaggacgggacgtgcagggtcacctactgccccacagagccaggcaactacatcatcaacatcaagtttgccgaccagcacgtgcctggcagccccttctctgtgaaggtgacaggcgagggccgggtgaaagagagcatcacccgcaggcgtcgggctccttcagtggccaacgttggtagtcattgtgacctcagcctgaaaatccctgaaattagcatccaggatatgacagcccaggtgaccagcccatcgggcaagacccatgaggccgagatcgtggaaggggagaaccacacctactgcatccgctttgttcccgctgagatgggcacacacacagtcagcgtgaagtacaagggccagcacgtgcctgggagccccttccagttcaccgtggggcccctaggggaagggggagcccacaaggtccgagctgggggccctggcctggagagagctgaagctggagtgccagccgaattcagtatctggacccgggaagctggtgctggaggcctggccattgctgtcgagggccccagcaaggctgagatctcttttgaggaccgcaaggacggctcctgtggtgtggcttatgtggtccaggagccaggtgactacgaagtctcagtcaagttcaacgaggaacacattcccgacagccccttcgtggtgcctgtggcttctccgtctggcgacgcccgccgcctcactgtttctagccttcaggagtcagggctaaaggtcaaccagccagcctcttttgcagtcagcctgaacggggccaagggggcgatcgatgccaaggtgcacagcccctcaggagccctggaggagtgctatgtcacagaaattgaccaagataagtatgctgtgcgcttcatccctcgggagaatggcgtttacctgattgacgtcaagttcaacggcacccacatccctggaagccccttcaagatccgagttggggagcctgggcatggaggggacccaggcttggtgtctgcttacggagcaggtctggaaggcggtgtcacagggaacccagctgagttcgtcgtgaacacgagcaatgcgggagctggtgccctgtcggtgaccattgacggcccctccaaggtgaagatggattgccaggagtgccctgagggctaccgcgtcacctatacccccatggcacctggcagctacctcatctccatcaagtacggcggcccctaccacattgggggcagccccttcaaggccaaagtcacaggcccccgtctcgtcagcaaccacagcctccacgagacatcatcagtgtttgtagactctctgaccaaggccacctgtgccccccagcatggggccccgggtcctgggcctgctgacgccagcaaggtggtggccaagggcctggggctgagcaaggcctacgtaggccagaagagcagcttcacagtagactgcagcaaagcaggcaacaacatgctgctggtgggggttcatggcccaaggaccccctgcgaggagatcctggtgaagcacgtgggcagccggctctacagcgtgtcctacctgctcaaggacaagggggagtacacactggtggtcaaatggggggacgagcacatcccaggcagcccctaccgcgttgtggtgccctgagtctggggcccgtgccagccggcagcccccaagcctgccccgctacccaagcagccccgccctcttcccctcaaccccggcccaggccgccctggccgcccgcctgtcactgcagccgcccctgccctgtgccgtgctgcgctcacctgcctccccagccagccgctgacctctcggctttcacttgggcagagggagccatttggtggcgctgcttgtcttctttggttctgggaggggtgagggatgggggtcctgtacacaaccacccactagttctcttctccagccaagaggaataaagttttgcttccattc

SEQ ID NO: 5 - DNA, 20 bp, Artificial Sequence
  Primer
ggatgtgactcaacctcccg

SEQ ID NO: 6 - DNA, 20 bp, Artificial Sequence
  Primer
cgggctacattcacccagag

SEQ ID NO: 7 - DNA, 20 bp, Artificial Sequence
  Primer
aagggggagtacacactggt

SEQ ID NO: 8 - DNA, 18 bp, Artificial Sequence
  Primer
caccacaacgcggtaggg

SEQ ID NO: 9 - DNA, 21 bp, Artificial sequence
  Sense strand of siRNA
gaaccuauaucgucaccautt

SEQ ID NO: 10 - RNA, 21 nt, Artificial sequence
  Antisense strand of siRNA
auggugacgauauagguucca

SEQ ID NO: 11 - DNA, 21 bp, Artificial sequence
  Sense strand of siRNA
ccaacaagcccaacaaguutt

SEQ ID NO: 12 - DNA, 21 bp, Artificial sequence
  Antisense strand of siRNA
aacuuguugggcuuguuggtg

SEQ ID NO: 13 - DNA, 21 bp, Artificial sequence
  Sense strand of siRNA
gcauucuagucaaguacaatt

SEQ ID NO: 14 - DNA, 21 bp, Artificial sequence
  Antisense strand of siRNA
uuguacuugacuagaaugctg

SEQ ID NO: 15 - DNA, 10031 bp, Artificial sequence
  Transgene
taatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattaaccctcactaaaggggtaccgggccccccctcgagccaccatgagtagctcccactctcgggcgggccagagcgcagcaggcgcggctccgggcggcggcgtcgacacgcgggacgccgagatgccggccaccgagaaggacctggcggaggacgcgccatggaagaagatccagcagaacactttcacgcgctggtgcaacgagcacctgaagtgcgtgagcaagcgcatcgccaacctgcagacggacctgagcgacgggctgcggcttatcgcgctgttggaggtgctcagccagaagaagatgcaccgcaagcacaaccagcggcccactttccgccaaatgcagcttgagaacgtgtcggtggcactcgagttcctggaccgcgagagcatcaaactggtgtccatcgacagcaaggccatcgtggacgggaacctgaagctgatcctgggcctcatctggaccctgatcctgcactactccatctccatgcccatgtgggacgaggaggaggatgaggaggccaagaagcagacccccaagcagaggctcctgggctggatccagaacaagctgccgcagctgcccatcaccaacttcagccgggactggcagagcggccgggccctgggcgccctggtggacagctgtgccccgggcctgtgtcctgactgggactcttgggacgccagcaagcccgttaccaatgcgcgagaggccatgcagcaggcggatgactggctgggcatcccccaggtgatcacccccgaggagattgtggaccccaacgtggacgagcactctgtcatgacctacctgtcccagttccccaaggccaagctgaagccaggggctcccttgcggcccaaactgaacccgaagaaagcccgtgcctacgggccaggcatcgagcccacaggcaacatggtgaagaagcgggcagagttcactgtggagaccagaagtgctggccagggagaggtgctggtgtacgtggaggacccggccggacaccaggaggaggcaaaagtgaccgccaataacgacaagaaccgcaccttctccgtctggtacgtccccgaggtgacggggactcataaggttactgtgctctttgctggccagcacatcgccaagagccccttcgaggtgtacgtggataagtcacagggtgacgccagcaaagtgacagcccaaggtcccggcctggagcccagtggcaacatcgccaacaagaccacctactttgagatctttacggcaggagctggcacgggcgaggtcgaggttgtgatccaggaccccatgggacagaagggcacggtagagcctcagctggaggcccggggcgacagcacataccgctgcagctaccagcccaccatggagggcgtccacaccgtgcacgtcacgtttgccggcgtgcccatccctcgcagcccctacactgtcactgttggccaagcctgtaacccgagtgcctgccgggcggttggccggggcctccagcccaagggtgtgcgggtgaaggagacagctgacttcaaggtgtacacaaagggcgctggcagtggggagctgaaggtcaccgtgaagggccccaagggagaggagcgcgtgaagcagaaggacctgggggatggcgtgtatggcttcgagtattaccccatggtccctggaacctatatcgtcaccatcacgtggggtggtcagaacatcgggcgcagtcccttcgaagtgaaggtgggcaccgagtgtggcaatcagaaggtacgggcctggggccctgggctggagggcggcgtcgttggcaagtcagcagactttgtggtggaggctatcggggacgacgtgggcacgctgggcttctcggtggaagggccatcgcaggctaagatcgaatgtgacgacaagggcgacggctcctgtgatgtgcgctactggccgcaggaggctggcgagtatgccgttcacgtgctgtgcaacagcgaagacatccgcctcagccccttcatggctgacatccgtgacgcgccccaggacttccacccagacagggtgaaggcacgtgggcctggattggagaagacaggtgtggccgtcaacaagccagcagagttcacagtggatgccaagcacggtggcaaggccccacttcgggtccaagtccaggacaatgaaggctgccctgtggaggcgttggtcaaggacaacggcaatggcacttacagctgctcctacgtgcccaggaagccggtgaagcacacagccatggtgtcctggggaggcgtcagcatccccaacagccccttcagggtgaatgtgggagctggcagccaccccaacaaggtcaaagtatacggccccggagtagccaagacagggctcaaggcccacgagcccacctacttcactgtggactgcgccgaggctggccagggggacgtcagcatcggcatcaagtgtgcccctggagtggtaggccccgccgaagctgacatcgacttcgacatcatccgcaatgacaatgacaccttcacggtcaagtacacgccccggggggctggcagctacaccattatggtcctctttgctgaccaggccacgcccaccagccccatccgagtcaaggtggagccctctcatgacgccagtaaggtgaaggccgagggccctggcctcagtcgcactggtgtcgagcttggcaagcccacccacttcacagtaaatgccaaagctgctggcaaaggcaagctggacgtccagttctcaggactcaccaagggggatgcagtgcgagatgtggacatcatcaaccaccatgacaacacctacacagtcaagtacacgcctgtccagcagggtccagtaggcgtcaatgtcacttatggaggggatcccatccctaagagccctttctcagtggcagtatctccaagcctggacctcagcaagatcaaggtgtctggcctgggagagaaggtggacgttggcaaagaccaggagttcacagtcaaatcaaagggtgctggtggtcaaggcaaagtggcatccaagattgtgggcccctcgggtgcagcggtgccctgcaaggtggagccaggcctgggggctgacaacagtgtggtgcgcttcctgccccgtgaggaagggccctatgaggtggaggtgacctatgacggcgtgcccgtgcctggcagcccctttcctctggaagctgtggcccccaccaagcctagcaaggtgaaggcgtttgggccggggctgcagggaggcagtgcgggctcccccgcccgcttcaccatcgacaccaagggcgccggcacaggtggcctgggcctgacggtggagggcccctgtgaggcgcagctcgagtgcttggacaatggggatggcacatgttccgtgtcctacgtgcccaccgagcccggggactacaacatcaacatcctcttcgctgacacccacatccctggctccccattcaaggcccacgtggttccctgctttgacgcatccaaagtcaagtgctcaggccccgggctggagcgggccaccgctggggaggtgggccaattccaagtggactgctcgagcgcgggcagcgcggagctgaccattgagatctgctcggaggcggggcttccggccgaggtgtacatccaggaccacggtgatggcacgcacaccattacctacattcccctctgccccggggcctacaccgtcaccatcaagtacggcggccagcccgtgcccaacttccccagcaagctgcaggtggaacctgcggtggacacttccggtgtccagtgctatgggcctggtattgagggccagggtgtcttccgtgaggccaccactgagttcagtgtggacgcccgggctctgacacagaccggagggccgcacgtcaaggcccgtgtggccaacccctcaggcaacctgacggagacctacgttcaggaccgtggcgatggcatgtacaaagtggagtacacgccttacgaggagggactgcactccgtggacgtgacctatgacggcagtcccgtgcccagcagccccttccaggtgcccgtgaccgagggctgcgacccctcccgggtgcgtgtccacgggccaggcatccaaagtggcaccaccaacaagcccaacaagttcactgtggagaccaggggagctggcacgggcggcctgggcctggctgtagagggcccctccgaggccaagatgtcctgcatggataacaaggacggcagctgctcggtcgagtacatcccttatgaggctggcacctacagcctcaacgtcacctatggtggccatcaagtgccaggcagtcctttcaaggtccctgtgcatgatgtgacagatgcgtccaaggtcaagtgctctgggcccggcctgagcccaggcatggttcgtgccaacctccctcagtccttccaggtggacacaagcaaggctggtgtggccccattgcaggtcaaagtgcaagggcccaaaggcctggtggagccagtggacgtggtagacaacgctgatggcacccagaccgtcaattatgtgcccagccgagaagggccctacagcatctcagtactgtatggagatgaagaggtaccccggagccccttcaaggtcaaggtgctgcctactcatgatgccagcaaggtgaaggccagtggccccgggctcaacaccactggcgtgcctgccagcctgcccgtggagttcaccatcgatgcaaaggacgccggggagggcctgctggctgtccagatcacggatcccgaaggcaagccgaagaagacacacatccaagacaaccatgacggcacgtatacagtggcctacgtgccagacgtgacaggtcgctacaccatcctcatcaagtacggtggtgacgagatccccttctccccgtaccgcgtgcgtgccgtgcccaccggggacgccagcaagtgcactgtcacaggtgctggcatcggccccaccattcagattggggaggagacggtgatcactgtggacactaaggcggcaggcaaaggcaaagtgacgtgcaccgtgtgcacgcctgatggctcagaggtggatgtggacgtggtggagaatgaggacggcactttcgacatcttctacacggccccccagccgggcaaatacgtcatctgtgtgcgctttggtggcgagcacgtgcccaacagccccttccaagtgacggctctggctggggaccagccctcggtgcagccccctctacggtctcagcagctggccccacagtacacctacgcccagggcggccagcagacttgggccccggagaggcccctggtgggtgtcaatgggctggatgtgaccagcctgaggccctttgaccttgtcatccccttcaccatcaagaagggcgagatcacaggggaggttcggatgccctcaggcaaggtggcgcagcccaccatcactgacaacaaagacggcaccgtgaccgtgcggtatgcacccagcgaggctggcctgcacgagatggacatccgctatgacaacatgcacatcccaggaagccccttgcagttctatgtggattacgtcaactgtggccatgtcactgcctatgggcctggcctcacccatggagtagtgaacaagcctgccaccttcaccgtcaacaccaaggatgcaggagaggggggcctgtctctggccattgagggcccgtccaaagcagaaatcagctgcactgacaaccaggatgggacatgcagcgtgtcctacctgcctgtgctgccgggggactacagcattctagtcaagtacaatgaacagcacgtcccaggcagccccttcactgcccgggtcacaggtgacgactccatgcgtatgtcccacctaaaggtcggctctgctgccgacatccccatcaacatctcagagacggatctcagcctgctgacggccactgtggtcccgccctcgggccgggaggagccctgtttgctgaagcggctgcgtaatggccacgtggggatttcattcgtgcccaaggagacgggggagcacctggtgcatgtgaagaaaaatggccagcacgtggccagcagccccatcccggtggtgatcagccagtcggaaattggggatgccagtcgtgttcgggtctctggtcagggccttcacgaaggccacacctttgagcctgcagagtttatcattgatacccgcgatgcaggctatggtgggctcagcctgtccattgagggccccagcaaggtggacatcaacacagaggacctggaggacgggacgtgcagggtcacctactgccccacagagccaggcaactacatcatcaacatcaagtttgccgaccagcacgtgcctggcagccccttctctgtgaaggtgacaggcgagggccgggtgaaagagagcatcacccgcaggcgtcgggctccttcagtggccaacgttggtagtcattgtgacctcagcctgaaaatccctgaaattagcatccaggatatgacagcccaggtgaccagcccatcgggcaagacccatgaggccgagatcgtggaaggggagaaccacacctactgcatccgctttgttcccgctgagatgggcacacacacagtcagcgtcaagtacaagggccagcacgtgcctgggagccccttccagttcaccgtggggcccctaggggaagggggagcccacaaggtccgagctgggggccctggcctggagagagctgaagctggagtgccagccgaattcagtatctggacccgggaagctggtgctggaggcctggccattgctgtcgagggccccagcaaggctgagatctcttttgaggaccgcaaggacggctcctgtggtgtggcttatgtggtccaggagccaggtgactacgaagtctcagtcaagttcaacgaggaacacattcccgacagccccttcgtggtgcctgtggcttctccgtctggcgacgcccgccgcctcactgtttctagccttcaggagtcagggctaaaggtcaaccagccagcctcttttgcagtcagcctgaacggggccaagggggcgatcgatgccaaggtgcacagcccctcaggagccctggaggagtgctatgtcacagaaattgaccaagataagtatgctgtgcgcttcatccctcgggagaatggcgtttacctgattgacgtcaagttcaacggtacccacatccctggaagccccttcaagatccgagttggggagcctgggcatggaggggacccaggcttggtgtctgcttacggagcaggtctggaaggcggtgtcacagggaacccagctgagttcgtcgtgaacacgagcaatgcgggagctggtgccctgtcggtgaccattgacggcccctccaaggtgaagatggattgccaggagtgccctgagggctaccgcgtcacctatacccccatggcacctggcagctacctcatctccatcaagtacggcggcccctaccacattgggggcagccccttcaaggccaaagtcacaggcccccgtctcgtcagcaaccacagcctccacgagacatcatcagtgtttgtagactctctgaccaaggccacctgtgccccccagcatggggccccgggtcctgggcctgctgacgccagcaaggtggtggccaagggcctggggctgagcaaggcctacgtaggccagaagagcagcttcacagtagactgcagcaaagcaggcaacaacatgctgctggtgggggttcatggcccaaggaccccctgcgaggagatcctggtgaagcacgtgggcagccggctctacagcgtgtcctacctgctcaaggacaagggggagtacacactggtggtcaaatggggggacgagcacatcccaggcagcccctaccgcgttgtggtgcccggtaccgagctcggatccgccaccgaattctgcagatatccagcacagtggcggccgctcgagtctagagggcccgattacaaggatgacgacgataagtgataaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagat